(19) United States
(12) Patent Application Publication
(10) Pub. No.: US 2010/0268087 A1
(43) Pub. Date: Oct. 21, 2010

(54) THREE-DIMENSIONAL IMAGE CONSTRUCTING APPARATUS AND IMAGE PROCESSING METHOD THEREOF

(75) Inventor: Kazuhiro HIROTA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/761,043

(22) Filed: Apr. 15, 2010

(30) Foreign Application Priority Data
Apr. 16, 2009 (JP) 2009-100166

(51) Int. Cl. A61B 8/14

(52) U.S. Cl. 600/445

(57) ABSTRACT

Three-dimensional image data is constructed without reducing precision of tomographic image data by each radial scanning which is acquired by scanning in a longitudinal axis direction of a probe even when a variation of rotational speed of radial scanning occurs due to a variation of torque in wave radiation from a probe distal end. A signal processing unit is configured by including an A/D conversion section, a line data generating section, a frame memory section, a memory control section, a data recording control section, an image constructing section, a data recording section, an longitudinal moving amount calculating section and a control section. The frame memory section stores reflection intensity data from the line data generating section by frame unit based on a rotation detection signal Sa, and is configured by including a first memory, a second memory and a third memory which are constituted of three frame memories for storing reflection intensity data of three frames.

### BACKGROUND OF THE INVENTION

1. Field of the Invention

The present invention relates to a three-dimensional image constructing apparatus and an image processing method thereof, and particularly relates to a three-dimensional image constructing apparatus and an image processing method which have a characteristic in construction of a three-dimensional image by tomographic images obtained by radial scanning.

2. Related Art

Conventionally, an image diagnostic apparatus has been widely used, which visualizes the tomographic image of a living body by inserting a probe into a body cavity, and radially scanning a biological tissue by a wave.

Examples of the image diagnostic apparatus include an intraductal ultrasound(IDUS) or intravascular ultrasound(IVUS) diagnostic apparatus which uses ultrasound as a wave, causes the ultrasound from an ultrasound transducer to scan radially, receives a reflected wave (ultrasound echo) reflected at the biological tissue in the body cavity with the same ultrasound transducer, thereafter, performs processing of amplification, wave detection and the like, and visualizes the cross-sectional image of the body cavity based on the intensity of the generated ultrasound echo (Japanese Patent Application Laid-Open No. 2003-310618).

Further, an intraductal ultrasound diagnostic apparatus is also used, which three-dimensionally acquires a tomographic image by causing the ultrasound transducer to scan in the longitudinal direction simultaneously with radial scanning (Japanese Patent Application Laid-Open No. 2000-116654). Japanese Patent Application Laid-Open No. 2000-116654 discloses the art of selecting data at constant intervals to construct the data as three-dimensional data by selecting only the images corresponding to pitch intervals based on the set number of unit images and capturing the images by the three-dimensional ultrasound image generating device, and thinning out the other ultrasound images (selecting a small number of images in accordance with the moving distance out of a large number of acquired images).

Further, in addition to the ultrasound diagnostic apparatus, an optical coherent tomography (OCT: Optical Coherent Tomography) with a light used as the wave has been used as the image diagnostic apparatus in recent years.

An optical coherent tomography divides a low coherent light into a measurement light and a reference light, inserts a probe containing an optical fiber with an optical lens and an optical mirror attached to a distal end into a body cavity, radiates the measurement light into a body cavity while causing the optical mirror disposed at the distal end side of the optical fiber to scan radially, and visualizes the cross-sectional image of the body cavity based on the coherent intensity of the reflected light from the tissue and the reference light (Japanese Patent Application Laid-Open No. 2007-268131).

For example, in an optical coherent tomography, as shown in FIG. 24, when the probe is inserted into a body cavity and biological tissue is radially scanned with a measurement light, a cross-sectional image perpendicular to the probe can be basically visualized. In contrast with this, when the measurement light is caused to scan biological tissue in the longitudinal axis direction of the probe simultaneously with radial scanning, the measurement light is actually caused to scan spirally as shown in FIG. 25. By constructing a approximate tomographic image of one frame at each radial scanning as shown in FIG. 26 by the spiral scanning, a plurality of tomographic image data at constant intervals in the longitudinal axis direction are generated. By arranging a plurality of these tomographic image data and handling them as three-dimensional data, three-dimensional analysis is enabled. FIG. 26 shows, for example, tomographic images which are constructed when longitudinal scanning is performed at 0.5 mm/sec while performing radial scanning at a rotational speed of 50 Hz (3000 rpm). The radial scanning speed and the longitudinal scanning speed are set at 50 Hz and 0.5 mm/sec respectively, but they are not especially limited to these values.

Generally when three-dimensional data is constructed by irradiating biological tissue with wave like this, the data acquisition and analysis are performed on the assumption that rotational speed and scanning speed are constant as set value. More specifically, the example of FIG. 26 adopts 50 frame/sec and 0.5 mm/sec, and therefore, the acquired tomographic data becomes the data of 10 μm/frame. More specifically, the distance in the longitudinal axis direction is expressed by frame unit.

However, when a probe is caused to scan radially, the rotational speed may temporarily varies due to torque variation and the like in accordance with the disposition state of the probe in a body cavity. In such a case, when the rotational speed reduces (for example, the rotational speed of radial scanning reduces to 40 Hz from 50 Hz) as shown in FIG. 27, the time in which the tomographic image of one frame is constructed becomes longer than assumed, and the moving distance in the longitudinal axis direction during this time becomes long. Therefore, the data obtained as a result are not such data that are at constant intervals in the longitudinal axis direction, and the distance precision in the longitudinal axis direction when the data is three-dimensionally analyzed is reduced.

Thus, the above described Japanese Patent Application Laid-Open No. 2003-310618 discloses the art which changes the rotational speed of radial scanning in accordance with the variation in the actual moving speed in the longitudinal axis direction by controlling the rotational speed of the ultrasound transducer to be propartal to the moving speed when a three-dimensional image is constructed while manually causing an ultrasound probe to scan, by providing a speed sensor at the ultrasound probe (ultra sound endoscope). More specifically, in Japanese Patent Application Laid-Open No. 2003-310618, the rotational speed of radial scanning is increased when the moving speed in the longitudinal axis direction becomes high, whereas when the moving speed in the longitudinal axis direction becomes low, the rotational speed of radial scanning is made low.

Further, the above described Japanese Patent Application Laid-Open 2000-116654 discloses the art in which a signal is outputted at each constant distance of longitudinal scanning, and from a number of tomographic image data obtained by longitudinal scanning, the tomographic image is selected in correspondence with the positional signals.

However, in the art of the above described Japanese Patent Application Laid-Open No. 2003-310618, since manual longitudinal scanning is the precondition, the precision of longitudinal scanning is extremely low with respect to radial scanning, under such a situation, the speed sensor is provided at the ultrasound probe (ultrasound endoscope), and by controlling the rotational speed of the ultrasound transducer to be propartal to the moving speed when constructing a three-dimensional image while manually causing the ultrasound probe to scan, whereby a three-dimensional image with few crude density parts is constructed irrespective of the scanning variation of an operator. At present, radial scanning and longitudinal scanningare both mechanically controlled in general, and longitudinal scanning is driven by using a ball screw, whereas radial scanning is driven by a DC motor. Therefore, mechanical precision of the longitudinal scanning is far higher than that of radial scanning, and there is the problem that controlling radial scanning with the precision of the longitudinal scanning which is higher than this is difficult.

Further, in the art of the above described Japanese Patent Application

Laid-Open No. 2000-116654, tomographic data is extracted in correspondence with the moving distance out of a number of acquired tomographic image data, and therefore, a large number of tomographic image data which are not used though acquired are present, which results in much waste in processing. Further, especially when three-dimensional data analysis is performed, there is the demand for acquiring tomographic images at the density as high as possible, but this art runs counter to this demand.

### SUMMARY OF THE INVENTION

The present invention is made in view of such circumstances, and has an object to provide a three-dimensional image constructing apparatus and an image processing method thereof which can construct three-dimensional image data without reducing precision of tomographic image data by each radial scanning acquired by scanning in a probe longitudinal axis direction even when a variation of rotational speed of radial scanning occurs due to a variation of torque in wave irradiation from a distal end of a probe.

In order to attain the aforementioned object, a three-dimensional image constructing apparatus according to a first aspect of the present invention is configured by including a wave transmitting/receiving device which is provided in a distal end of a slim and substantially tubular probe having flexibility, and transmits and receives a wave, a transmission/reception wave rotating device which rotates the wave transmitting/receiving device around a longitudinal axis of the probe and causes the wave to scan radially on a scan surface including a depth direction of a measuring object, a rotation detecting device which detects rotation of the transmission/reception wave rotating device and outputs a rotation detection signal, a tomographic information generating device which generates tomographic information of the measuring object from reflection wave information of the wave which is caused to scan radially and is reflected at the measuring object, based on the rotation detection signal from the rotation detecting device, a tomographic information storing device which stores the tomographic information by frame unit, a storage control device which controls write and read of tomographic information in the tomographic information storing device, a transmission/reception wave moving device which moves the wave transmitting/receiving device along the longitudinal axis direction, an evenly spaced tomographic image generating device which generates an evenly spaced tomographic image of the measuring object at a moving position at each of constant equal spaces along the longitudinal axis direction by the transmission/reception wave moving device, based on the tomographic information which is read from the tomographic information storing device by being controlled by the storage control device, and a three-dimensional image generating device which generates a three-dimensional image of the measuring object based on the evenly spaced tomographic image.

In the three-dimensional image constructing apparatus according to the first aspect, by the transmission/reception wave rotating device, the wave transmitting/receiving device is rotated around a longitudinal axis of the probe, and the wave is caused to scan radially on a scan surface including a depth direction of a measuring object, rotation of the transmission/reception wave rotating device is detected and a rotation detection signal is outputted by the rotation detecting device, tomographic information of the measuring object is generated from reflection wave information of the wave which is caused to scan radially and is reflected at the measuring object, based on the rotation detection signal from the rotation detecting device by the tomographic information generating device, the tomographic information is stored by frame unit by the tomographic information storing device, write and read of tomographic information in the tomographic information storing device is controlled by the storage control device, the wave transmitting/receiving device is moved along the longitudinal axis direction by the transmission/reception wave moving device, an evenly spaced tomographic image of the measuring object at a moving position at each of constant equal spaces along the longitudinal axis direction by the transmission/reception wave moving device is generated, based on the tomographic information which is read from the tomographic information storing device by being controlled by the storage control device in the evenly spaced tomographic image generating device, and a three-dimensional image of the measuring object is generated based on the evenly spaced tomographic image by the three-dimensional image generating device. Thereby, even when a variation of the rotational speed of radial scanning occurs due to a variation of torque in wave irradiation from a probe distal end, a three-dimensional image data can be constructed without reducing precision of the tomographic image data by each radial scanning which is acquired by longitudinal scanning of the probe.

As in the three-dimensional image constructing apparatus according to a second aspect of the present invention, the three-dimensional image constructing apparatus according to the first aspect preferably further includes a first moving distance signal outputting device which estimates a moving distance of the wave transmitting/receiving device by the transmission/reception wave moving device in the longitudinal axis direction based on a time interval which is set in advance, and outputs a moving distance signal, and the storage control device preferably writes the tomographic information into the tomographic information storing device synchronously with an output time of the rotation detection signal, and reads the tomographic information stored in the tomographic information storing device synchronously with an output time of the moving distance signal.

As the three-dimensional image constructing apparatus according to a third aspect of the present invention, the three-dimensional image constructing apparatus according to the first aspect preferably further includes a second moving distance signal outputting device which detects a moving distance of the wave transmitting/receiving device in the longitudinal axis direction, and outputs a moving distance signal, and the storage control device preferably writes the tomographic information into the tomographic information storing device synchronously with an output time of the rotation detection signal, and reads the tomographic information stored in the tomographic information storing device synchronously with an output time of the moving distance signal.

As the three-dimensional image constructing apparatus according to a fourth aspect of the present invention, in the three-dimensional image constructing apparatus according to any one of the first to the third aspects, the tomographic information storing device is preferably constituted of a plurality of frame memories which store the tomographic information of a plurality of frames.

As the three-dimensional image constructing apparatus according to a fifth aspect of the present invention, in the three-dimensional image constructing apparatus according to the fourth aspect, the storage control device preferably stores the tomographic information which is newly generated by the tomographic information generating device in the frame memory which stores the earliest tomographic image in a sequence of generation by the tomographic information generating device, among the frame memories in which read processing is not performed in the tomographic information storing device, and reads the tomographic information from the frame memory which stores the latest tomographic information in the sequence of generation by the tomographic information generating device among the frame memories in which write processing is not performed in the tomographic information storing device.

As the three-dimensional image constructing apparatus according to a sixth aspect of the present invention, in the three-dimensional image constructing apparatus according to the fourth or the fifth aspect, the tomographic information storing device is preferably constituted of three frame memories which store the tomographic information of at least three frames.

As the three-dimensional image constructing apparatus according to a seventh aspect of the present invention, the three-dimensional image constructing apparatus according to any one of the first to the sixth aspects preferably further includes a time detecting device which detects a time of an output time of the rotation detection signal as first time information, and a time of an output time of the moving distance signal as second time information, a linking device which links the tomographic information generated by the tomographic information generating device, and the first time information and the second time information, and a time-added tomographic information storing device which stores the tomographic information to which the first time information and the second time information are linked in the linking device as time-added tomographic information.

As the three-dimensional image constructing apparatus according to an eighth aspect of the present invention, the three-dimensional image constructing apparatus according to the seventh aspect preferably further includes a real time clock having absolute time information, and the time detecting device preferably detects the first time information and the second time information based on the absolute time information of the real time clock.

As the three-dimensional image constructing apparatus according to a ninth aspect of the present invention, in the three-dimensional image constructing apparatus according to the seventh aspect, the time detecting device preferably detects a relative time with a detection time of the first time information as a reference, as the second time information.

As the three-dimensional image constructing apparatus according to a tenth aspect of the present invention, the three-dimensional image constructing apparatus according to any one of the seventh to the ninth aspects preferably further includes a tomographic image interpolating and generating device which interpolates the tomographic information and generates the evenly spaced tomographic image, based on the first time information and the second time information in accordance with a plurality of pieces of time-added tomographic information stored in the time-added tomographic information storing device.

As the three-dimensional image constructing apparatus according to an eleventh aspect of the present invention, in the three-dimensional image constructing apparatus according to any one of the first to the tenth aspects, the transmission/reception wave rotating device is preferably a flexible shaft with the longitudinal axis provided in the probe including the wave transmitting/receiving device at a distal end as a rotation axis, and the transmission/reception wave moving device preferably moves the flexible shaft along the longitudinal axis.

As the three-dimensional image constructing apparatus according to a twelfth aspect of the present invention, in the three-dimensional image constructing apparatus according to any one of the first to the eleventh aspects, it is preferable that the wave is a light, and the light is divided into a measurement light and a reference light, the probe is connected to a light source which outputs the light, through the optical rotary joint, and capable of transmitting and receiving the measurement light, and the tomographic information generating device generates the tomographic information by the frame unit based on a coherent light of a reflection light of the measurement light in a body cavity acquired by the probe and the reference light reflected in a predetermined path.

As the three-dimensional image constructing apparatus according to a thirteenth aspect of the present invention, in the three-dimensional image constructing apparatus according to the twelfth aspect, the light source is preferably a wavelength swept laser light source.

As the three-dimensional image constructing apparatus according to a fourteenth aspect of the present invention, in the three-dimensional image constructing apparatus according to any one of the first to the eleventh aspects, it is preferable that the wave is ultrasound, the probe includes an ultrasound transducer capable of transmitting and receiving the ultrasound, and the tomographic information generating device generates the tomographic information by the frame unit based on an echo signal of the ultrasound in the body cavity which is acquired by the probe.

An image processing method of a three-dimensional image constructing apparatus according to a fifteenth aspect of the present invention is configured by including a transmission/reception wave rotating step of rotating a wave transmitting/receiving device, which is provided in a distal end of a slim and substantially tubular probe having flexibility and transmits and receives a wave, around a longitudinal axis of the probe, and causing the wave to scan radially on a scan surface including a depth direction of a measuring object, a rotation detecting step of detecting rotation in the transmission/reception wave rotating step, and outputting a rotation detection signal, a tomographic information generating step of generating tomographic information of the measuring object from reflection wave information of the wave which is caused to scan radially and reflected at the measuring object, based on the rotation detection signal from the rotation detecting step, a tomographic information storing step of storing the tomographic information by frame unit, a storage control step of controlling write and read of tomographic information in the tomographic information storing step, a transmission/reception wave moving step of moving the wave transmitting/receiving device along the longitudinal axis direction, an evenly spaced tomographic image generating step of generating an evenly spaced tomographic image of the measuring object at a moving position at each of constant equal spaces along the longitudinal direction by the transmission/reception wave moving step, based on the tomographic information which is read from a tomographic information storing step by being controlled by the storage control step, and a three-dimensional image generating step of generating a three-dimensional image of the measuring object based on the evenly spaced tomographic image.

In the image processing method of the three-dimensional image constructing apparatus according to the fifteenth aspect, in the transmission/reception wave rotating step, the wave transmitting/receiving device is rotated around a longitudinal axis of the probe, and the wave is caused to scan radially on a scan surface including a depth direction of a measuring object, rotation of the transmission/reception wave rotating device is detected and a rotation detection signal is outputted in the rotation detecting step, tomographic information of the measuring object is generated from reflection wave information of the wave which is caused to scan radially and reflected at the measuring object, based on the rotation detection signal from the rotation detecting step in the tomographic information generating step, the tomographic information is stored by frame unit in the tomographic information storing step, write and read of tomographic information in the tomographic information storing step is controlled in the storage control step, the wave transmitting/receiving device is moved along the longitudinal axis direction in the transmission/reception wave moving step, an evenly spaced tomographic image of the measuring object at a moving position at each of constant equal spaces along the longitudinal axis direction by the transmission/reception wave moving step is generated, based on the tomographic information which is read from the tomographic information storing step by being controlled by the storage control step, in the evenly spaced tomographic image generating step, and a three-dimensional image of the measuring object is generated based on the evenly spaced tomographic image in the three-dimensional image generating step. Thereby, even when a variation of the rotational speed of radial scanning due to a variation of torque in wave irradiation from a probe distal end occurs, three-dimensional image data can be constructed without reducing precision of the tomographic image data by each radial scanning which is acquired by longitudinal scanning of the probe.

As the image processing method of the three-dimensional image constructing apparatus according to a sixteenth aspect of the present invention, the image processing method of the three-dimensional image constructing apparatus according to the fifteenth aspect preferably further includes a first moving distance signal outputting step of estimating a moving distance of the wave transmitting/receiving device by a transmission/reception wave moving step in the longitudinal axis direction based on a time interval which is set in advance, and outputting a moving distance signal, and it is preferable that in the storage control step, the tomographic information is written in the tomographic information storing step synchronously with an output time of the rotation detection signal, and the tomographic information is read from the tomographic information storing step synchronously with an output time of the moving distance signal.

As the image processing method of the three-dimensional image constructing apparatus according to a seventeenth aspect of the present invention, the image processing method of the three-dimensional image constructing apparatus according to the fifteenth aspect preferably further includes a second moving distance signal outputting step of detecting a moving distance in the wave transmitting/receiving moving step in the longitudinal axis direction, and outputting a moving distance signal, and it is preferable that in the storage control step, the tomographic information is written in the tomographic information storing step synchronously with an output time of the rotation detection signal, and the tomographic information is read from the tomographic information storing step synchronously with an output time of the moving distance signal.

As the image processing method of the three-dimensional image constructing apparatus according to an eighteenth aspect of the present invention, the image processing method of the three-dimensional image constructing apparatus according to any one of the fifteenth to the seventeenth aspects preferably further includes a time detecting step of detecting a time of an output time of the rotation detection signal as first time information, and a time of an output time of the moving distance signal as second time information, a linking step of linking the tomographic information generated in the tomographic information generating step, and the first time information and the second time information, and a time-added tomographic information storing step of storing the tomographic information to which the first time information and the second time information are linked in the linking step as time-added tomographic information.

As the image processing method of the three-dimensional image constructing apparatus according to a nineteenth aspect of the present invention, in the image processing method of the three-dimensional image constructing apparatus according to the eighteenth aspect, it is preferable that in the time detecting step, the first time information and the second time information are detected based on the absolute time information from the real time clock.

As the image processing method of the three-dimensional image constructing apparatus according to a twentieth aspect of the present invention, in the image processing method of the three-dimensional image constructing apparatus according to the eighteenth aspect, it is preferable that in the time detecting step, a relative time with a detection time of the first time information as a reference is detected as the second time information.

As the image processing method of the three-dimensional image constructing apparatus according to a twenty-first aspect of the present invention, the image processing method of the three-dimensional image constructing apparatus according to any one of the eighteenth to the twentieth aspects preferably further includes a tomographic image interpolating and generating step of interpolating the tomographic information and generating the evenly spaced tomographic image, based on the first time information and the second time information in accordance with a plurality of pieces of time-added tomographic information stored in the time-added tomographic information storing step.

As described above, according to the present invention, the effect is provided, that can construct three-dimensional image data without reducing precision of tomographic image data by each radial scanning acquired by longitudinal scanning of the probe even when a variation of rotational speed of radial scanning occurs due to a variation of torque in wave irradiation from a probe tip end.

### BRIEF DESCRIPTION OF THE DRAWINGS

FIG. 1 is an exterior view showing an image diagnostic apparatus according to a first embodiment of the present invention;

FIG. 2 is a block diagram showing an internal configuration of an OCT processor of FIG. 1;

FIG. 3 is a cross-sectional view showing a distal end section in a longitudinal axis direction of an OCT probe of FIG. 1;

FIG. 4 is a cross-sectional view showing a configuration of an optical rotary joint connecting a rotation side optical fiber FB1 of FIG. 3;

FIG. 5 is a view showing a state of obtaining optical structure information by using the OCT probe led out from a forceps channel of an endoscope of FIG. 1;

FIG. 6 is a block diagram showing a configuration of a signal processing unit of FIG. 2;

FIG. 7 is a first diagram for explaining a general operation of a frame memory section and a data recording control section of FIG. 6;

FIG. 8 is a second diagram for explaining a general operation of the frame memory section and the data recording control section of FIG. 6;

FIG. 9 is a third diagram for explaining a general operation of the frame memory section and the data recording control section of FIG. 6;

FIG. 10 is a flowchart showing a flow of a process of the signal processing unit of FIG. 6;

FIG. 11 is a timing chart showing a timing of a signal of the frame memory section in the process of FIG. 10;

FIG. 12 is a block diagram showing a modified example of the signal processing unit of FIG. 6;

FIG. 13 is a block diagram showing a configuration of an OCT processor according to a second embodiment of the present invention;

FIG. 14 is a block diagram of a signal processing unit of FIG. 13;

FIG. 15 is a block diagram of a signal processing unit according to a third embodiment of the present invention;

FIG. 16 is a flowchart showing a flow of a process of the signal processing unit of FIG. 15;

FIG. 17 is a timing chart showing a timing of a signal of a frame memory section in the process of FIG. 16;

FIG. 18 is a diagram explaining a processing result of FIG. 16;

FIG. 19 is a block diagram of a signal processing unit according to a fourth embodiment of the present invention;

FIG. 20 is a timing chart showing a timing of a signal of a frame memory section in a process of FIG. 19;

FIG. 21 is a block diagram showing a configuration of an ultrasound observation apparatus according to a fifth embodiment of the present invention;

FIG. 22 is a block diagram showing a configuration of a signal processing unit of FIG. 21;

FIG. 23 is a block diagram showing a configuration of a signal processing unit of an ultrasound observation apparatus according to a sixth embodiment of the present invention;

FIG. 24 is a view explaining radial scanning of a wave by a probe;

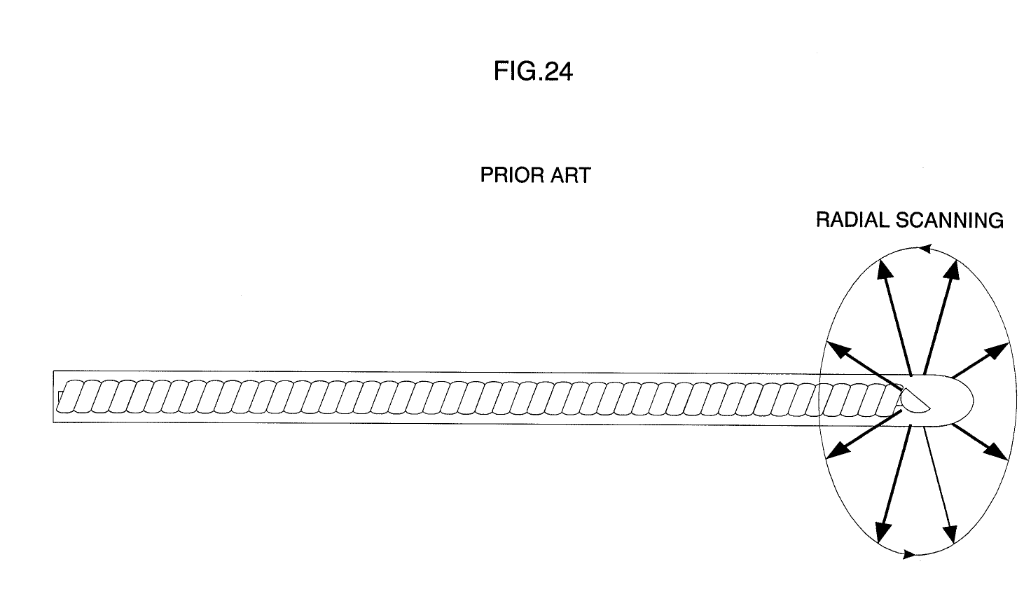

FIG. 25 is a view explaining spiral scanning of a wave by a probe;

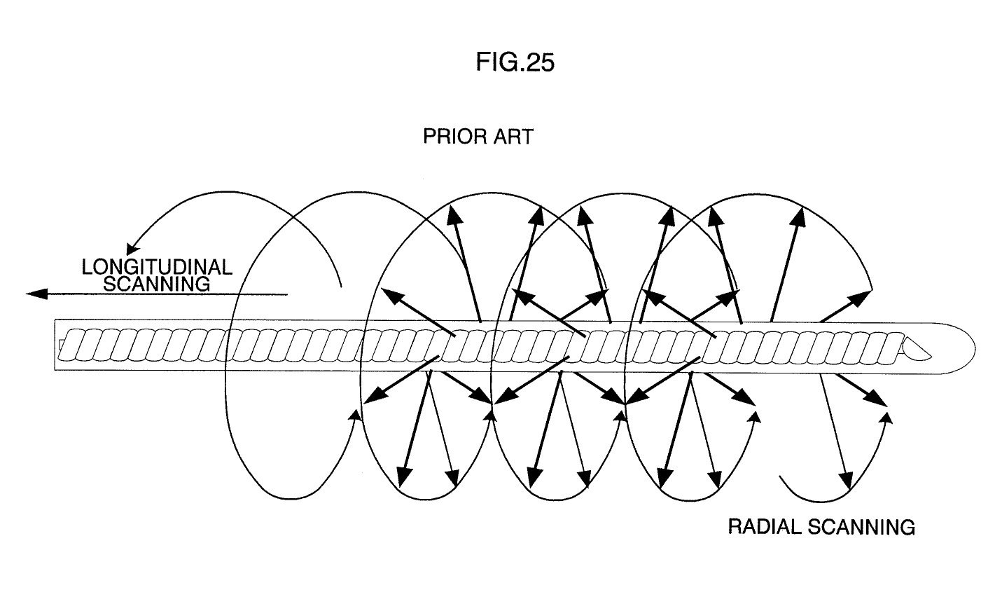

FIG. 26 is a diagram explaining tomographic image generation when radial scanning of the probe is stably performed; and

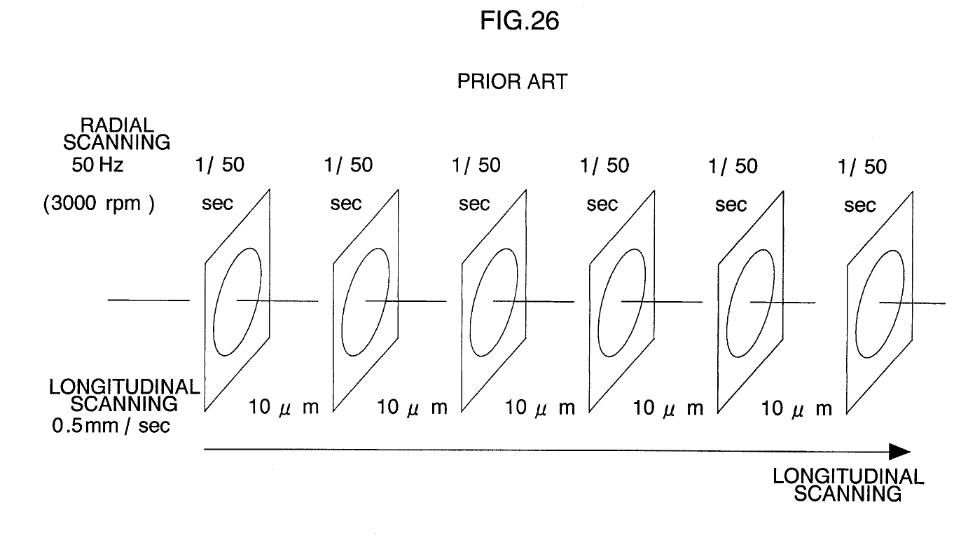

FIG. 27 is a diagram explaining tomographic image generation when a rotational speed of radial scanning of the probe is unstable.

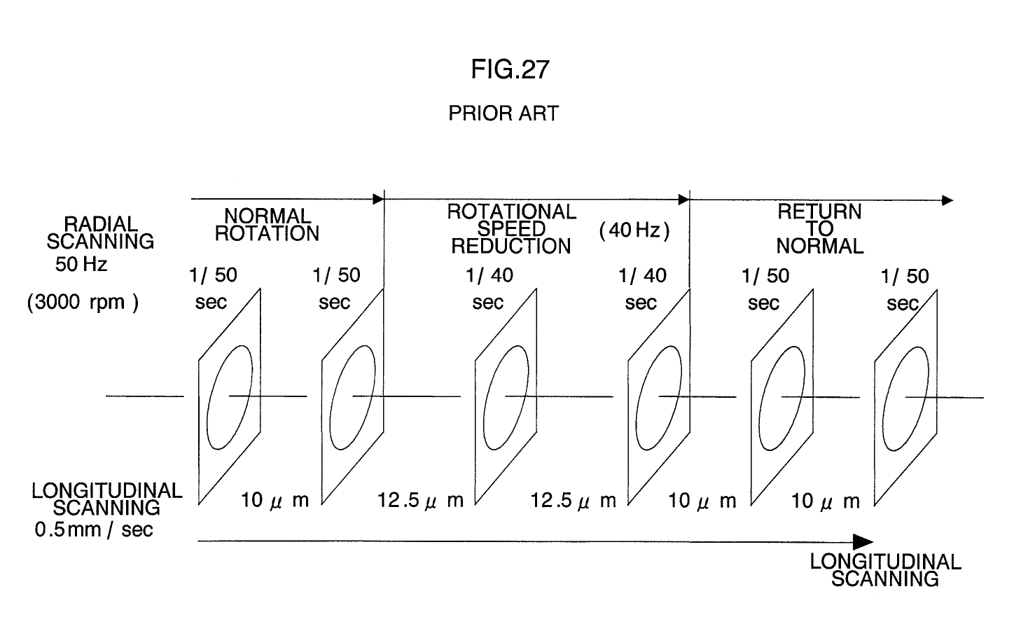

### DETAILED DESCRIPTION OF THE PREFERRED EMBODIMENTS

Hereinafter, embodiments of a three-dimensional image constructing apparatus according to the present invention will be described in detail with reference to the attached drawings.

### First Embodiment

<Appearance of Image Diagnostic Apparatus>

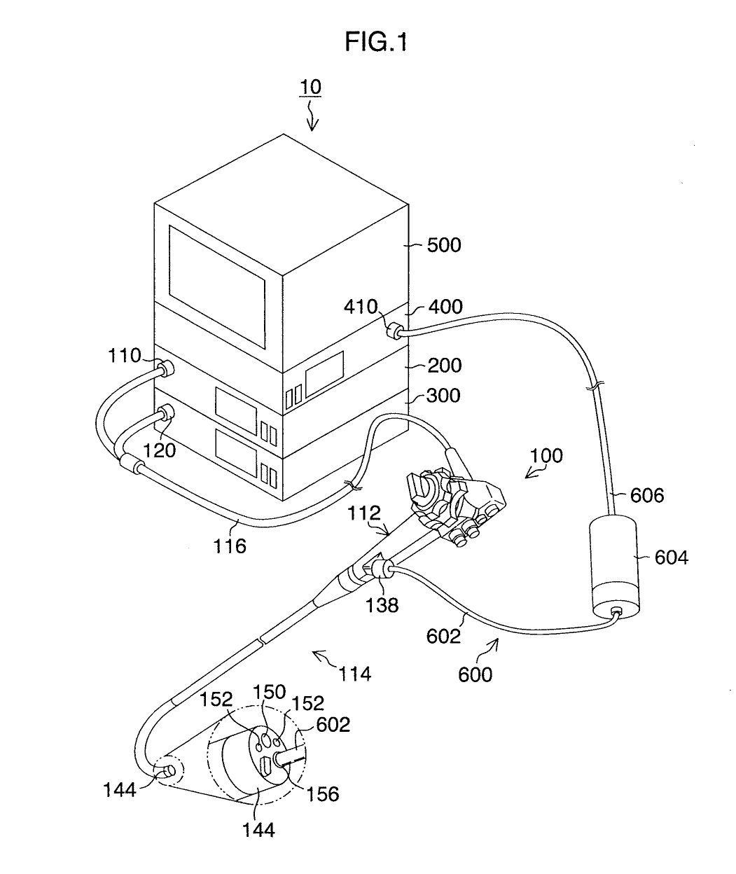

FIG. 1 is an external view showing an image diagnostic apparatus according to a first embodiment of the present invention.

As shown in FIG. 1, an image diagnostic apparatus 10 of the present embodiment is configured mainly by an endoscope 100, an endoscope processor 200, a light source device 300, an OCT processor 400 as a three-dimensional image constructing apparatus, and an image display unit 500 which is a monitor device. The endoscope processor 200 may be configured to contain the light source device 300 therein.

The endoscope 100 includes a hand operation part 112, and an insertion part 114 which is provided connectively to the hand operation part 112. An operator performs operation by grasping the hand operation part 112, and performs observation by inserting the insertion part 114 into the body of a subject.

The hand operation part 112 is provided with a forceps channel 138, and the forceps channel 138 communicates with a forceps channel 156 at a distal end part 144 through a forceps channel (not illustrated) provided inside the insertion part 114. In the image diagnostic apparatus 10, an OCT probe 600 as a probe is inserted from the forceps channel 138, and thereby, the OCT probe 600 is led out from the forceps channel 156. The OCT probe 600 is configured by an insertion part 602 which is inserted from the forceps channel 138 and is led out from the forceps channel 156, an operation part 604 for an operator to operate the OCT probe 600, and a cable 606 which is connected to the OCT processor 400 through a connector 410.

<Configurations of Endoscope, Endoscope Processor and Light source device>

[Endoscope]

At the distal end part 144 of the endoscope 100, an observation optical system 150, an illumination optical system 152, and a CCD (not illustrated) are placed.

The observation optical system 150 forms an image of a subject on a light receiving surface of the CCD not illustrated, and the CCD converts the subject image which is formed on the light receiving surface into an electric signal by each of light receiving elements. The CCD of this embodiment is a color CCD in which color filters of three primary colors of red (R), green (G) and blue (B) are placed in predetermined arrangements (Bayer array, honeycomb array) for each of pixels.

[Light Source Device]

The light source device 300 causes a visible light to be incident on a light guide (internally inserted in a cable 116 of the endoscope 100) not illustrated. One end of the light guide is connected to the light source device 300 through an LG connector 120, and the other end of the light guide faces the illumination optical system 152. The light emitted from the light source device 300 is radiated from the illumination optical system 152 through the light guide, and lights the visual field range of the observation optical system 150.

[Endoscope Processor]

An image signal which is outputted from the CCD through the cable 116 of the endoscope 100 is inputted into the endoscope processor 200 through an electric connector 110. This analog image signal is converted into a digital image signal in the endoscope processor 200, and is subjected to processing necessary for being displayed on the screen of the image display unit 500.

In this manner, the data of the observed image obtained by the endoscope 100 is outputted to the endoscope processor 200 and the image is displayed on the image display unit 500 connected to the endoscope processor 200.

<Internal Configurations of OCT Processor and OCT Probe>

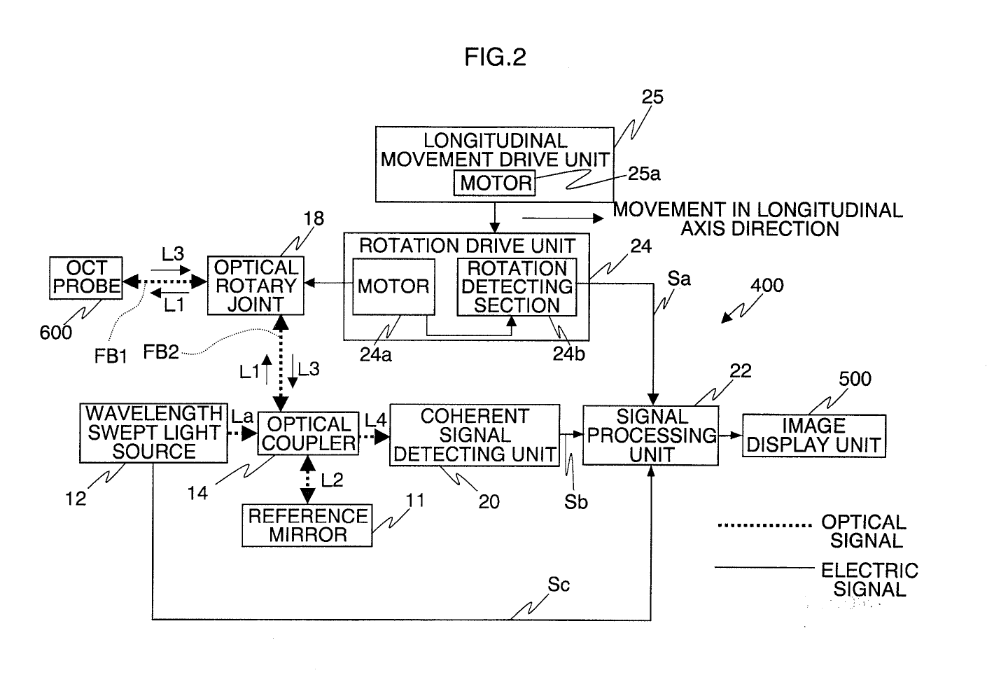

FIG. 2 is a block diagram showing an internal configuration of the OCT processor of FIG. 1.

[OCT Processor]

Next, an OCT processor of a first embodiment will be described by using FIG. 2. The OCT processor 400 is for acquiring an optical tomographic image of a measuring object by an optical coherence tomography (OCT: Optical Coherence Tomography) measurement method, and has a wavelength swept light source 12 which radiates a light La for measurement, an optical coupler 14 which divides the light La radiated from the wavelength swept light source 12 into a measurement light L1 and a reference light L2, and multiplexes a return light L3 from a measuring object S which is a specimen and the reference light L2 reflected by a reference mirror 11 to generate a coherent light L4, a rotation side optical fiber FB1 which guides the measurement light L1 divided by the optical coupler 14 to the measuring object and guides the return light L3 from the measuring object, and is included in the OCT probe 600, a fixed side optical fiber FB2 which guides the measurement light L1 to the rotation side optical fiber FB1 and guides the return light L3 guided by the rotation side optical fiber FB1, an optical rotary joint 18 which rotatably connects the rotation side optical fiber FB1 to the fixed side optical fiber FB2 and transmits the measurement light L1 and the return light L3, a coherent signal detecting unit 20 which detects the coherent light L4 which is generated by the optical coupler 14 as a coherent signal, and a signal processing unit 22 which processes a coherent signal Sb detected by the coherent signal detecting unit 20 and acquires optical structure information. Further, the image which is generated based on the optical structure information acquired in the signal processing unit 22 is displayed on the image display unit 500.

In the OCT processor 400 shown in FIG. 2, various optical fibers (not illustrated) including the rotation side optical fiber FB1 and the fixed side optical fiber FB2 are used as the optical paths for guiding and transmitting various lights including the aforementioned radiated light La, measurement light L1, reference light L2, return light 13 and the like among the components of each lighting device.

The wavelength swept light source 12 irradiates the light (for example, a laser light with a wavelength of 1.3 μm or a low coherence light) for measurement of OCT, and the wavelength swept light source 12 is a light source which radiates the laser light La with a wavelength of, for example, 1.3 μm which is in an infrared region as a center while sweeping the frequency at constant periods. The wavelength swept light source 12 includes a light source unit which radiates a laser light or the low coherence light La, and a lens which gathers the light La radiated from the light source unit, though not illustrated. Further, the light La is divided into the measurement light L1 and the reference light L2 by the optical coupler 14, and the measurement light L1 is inputted in the optical rotary joint 18. The wavelength swept light source 12 outputs a wavelength sweep synchronizing signal Sc synchronized with the period of wavelength sweep to the signal processing unit 22.

The optical rotary joint 18 guides the measurement light L1 to the rotation side optical fiber FB1 in the OCT probe 600.

The optical coupler 14 divides the light La from the wavelength swept light source 12 into the measurement light L1 and the reference light L2, causes the measurement light L1 to be incident on the fixed side optical fiber FB2, and causes the reference light L2 to be incident on the reference mirror 11 which adjusts the optical path length.

Further, the optical coupler 14 multiplexes the reference light L2 which is subjected to change of the optical path length by the reference mirror 11 and returns, and the return light L3 which is acquired by the OCT probe 600 which will be described later and is guided from the fixed side optical fiber FB2 to generate the coherent light L4 and outputs the coherent light L4 to the coherent signal detecting unit 20.

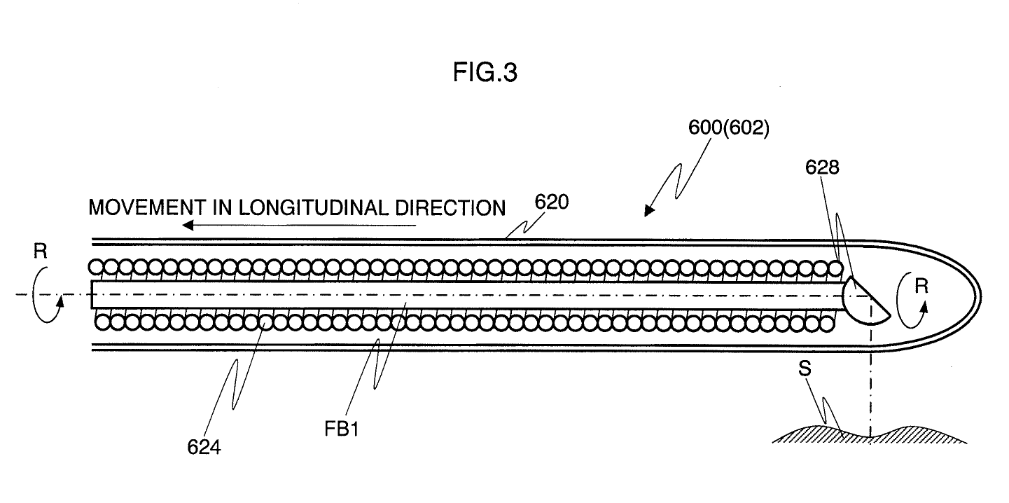

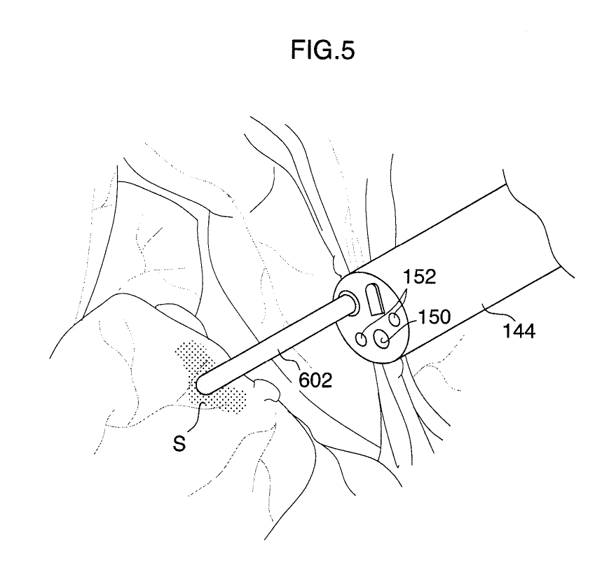

The OCT probe 600 is connected to the fixed side optical fiber FB2 through the optical rotary joint 18. The measurement light L1 is incident on the rotation side optical fiber FB1 from the fixed side optical fiber FB2 through the optical rotary joint 18, and the OCT probe 600 transmits the measurement light L1 by the rotation side optical fiber FB1 and irradiates the measuring object S with the measurement light L1 (see FIGS. 3 and 5). Next, the OCT probe 600 acquires the return light L3 from the measuring object S, transmits the acquired return light L3 by the rotation side optical fiber FB1, and radiates the return light L3 to the fixed side optical fiber FB2 through the optical rotary joint 18.

The coherent signal detecting unit 20 detects the coherent light L4 which is generated by multiplexing the reference light L2 and the return light L3 by the optical fiber coupler 14 as the coherent signal Sb, and the signal processing unit 22 at the next stage performs fast Fourier transform (FFT) of the coherent signal, and thereby, detects the intensity (optical structure information) of the reflected light (or backscattered light) in each depth position of the measuring object S.

More specifically, the signal processing unit 22 acquires the optical structure information from the coherence signal detected by the coherent signal detecting unit 20, generates an optical three-dimensional structure image based on the acquired optical structure image, and outputs the image which is obtained by applying various kinds of processing to the optical three-dimensional structure image to the image display unit 500. The detailed configuration of the signal processing unit 22 will be described later.

The reference mirror 11 is disposed at the radiation side of the reference light L2, makes the reference light L2 parallel light to gather it on the mirror and reflects the reference light L2 by the mirror. The mirror moves in the direction parallel with the optical axis direction by a mirror moving mechanism and thereby, adjusts the optical path length of the reference light L2.

The optical rotary joint 18 is controlled by a rotation drive unit 24 as a transmission/reception wave rotating device for performing radial scanning of the measurement light L1 from the rotation side optical fiber FB1 in the OCT probe 600, and an longitudinal movement drive unit 25 as a transmission/reception wave moving device for performing advance/retreat scanning along the longitudinal axis of the OCT probe 600.

In more detail, the rotation drive unit 24 is configured by including a motor 24a which rotationally drives the rotation side optical fiber FB1, and a rotation detecting section 24b as a rotation detecting device which outputs a pulse signal Sa of one pulse (one pulse/rotation) at each rotation of the motor 24a to the signal processing unit 22. Further, the longitudinal movement drive unit 25 includes a motor 25a, and performs advance/retreat scanning of the rotation side optical fiber FB1, the optical rotary joint 18 and the rotation drive unit 24 along the longitudinal axis of the OCT probe 600 by this motor 25a. The optical rotary joint 18 and the rotation drive unit 24 are provided in the operation unit 604 (see FIG. 1).

[OCT Probe]

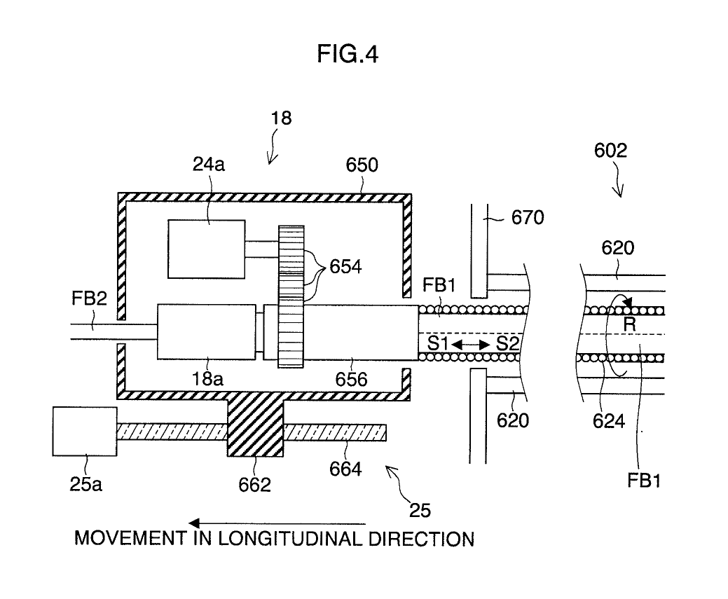

FIG. 3 is a cross-sectional view showing a distal end section in the longitudinal axis direction of the OCT probe of FIG. 1. Further, FIG. 4 is a cross-sectional view showing a configuration of an optical rotary joint to which the rotation side optical fiber FB1 of FIG. 3 is connected.

As shown in FIG. 3, in the OCT probe 600, a distal end part of an insertion part 602 has a substantially tubular sheath 620 with a distal end closed, the rotation side optical fiber FB1, a torque transmission coil 624 and an optical lens 628 as a wave transmitting/receiving device.

The sheath 620 is a tubular member having flexibility, and is formed from a material which allows the measurement light L1 and the return light L3 to pass through. In the sheath 620, a part at a side of a distal end (a distal end of the rotation side optical fiber FB1 at a side opposite from the optical rotary joint 18, hereinafter, called a distal end of the sheath 620) where the measurement light L1 and the return light L3 pass can be formed by a material (transparent material) which allows a light to pass through over the entire circumference, and a distal end part disposed at a distal end of the sheath 620 is formed into a substantially spherical shape in order to gather the measurement light L1 radiated from the rotation side optical fiber FB1 onto the measuring object S.

The optical lens 628 irradiates the measuring object S with the measurement light L1 radiated from the rotation side optical fiber FB1, and gathers the return light L3 from the measuring object S to cause the return light L3 to be incident on the rotation side optical fiber FB1.

Further, the rotation side optical fiber FB1 and the torque transmission coil 624 are connected to a rotary barrel 656 which will be described later, and the rotation side optical fiber FB1 and the torque transmission coil 623 are rotated by the rotary barrel 656, whereby the optical lens 628 is rotated in the arrow R direction with respect to the sheath 620. As shown in FIG. 4, the rotation side optical fiber FB1 and the fixed side optical fiber FB2 are connected by an optical connector 18a, and are optically connected in the state in which rotation of the rotation side optical fiber FB1 is not transmitted to the fixed side optical fiber FB2. Further, the rotation side optical fiber FB1 is disposed in the state rotatable with respect to the sheath 620 and movable in the longitudinal direction of the sheath 620.

The torque transmission coil 624 is fixed to the outer periphery of the rotation side optical fiber FB1. Further, the rotation side optical fiber FB1 and the torque transmission coil 624 are connected to the optical rotary joint 18.

Further, the rotation side optical fiber FB1, the torque transmission coil 624 and the optical lens 628 are configured to be movable in the arrow S1 direction (forceps channel direction) and the arrow S2 direction (direction of the distal end of the sheath 620) inside the sheath 620 by the advance/retreat drive unit which is provided in the optical rotary joint 18 and will be described later.

The sheath 620 is fixed to a fixed member 670. In contrast with this, the rotation side optical fiber FB1 and the torque transmission coil 624 are connected to the rotary barrel 656, and the rotary barrel 656 is configured to rotate in response to rotation of the motor 24a via a gear 654. The rotary barrel 656 is connected to the optical connector 18a of the optical rotary joint 18, and the measurement light L1 and the return light L3 are transmitted between the rotation side optical fiber FB1 and the fixed side optical fiber FB2 through the optical connector 18a.

Further, a frame 650 containing them therein is equipped with a support member 662, and the support member 662 has a screw hole not illustrated. In the optical rotary joint 18, an advancing/retreating ball screw 664 is meshed with the screw hole, a motor 25a is connected to the advancing/retreating ball screw 664, and the longitudinal movement drive unit 25 is configured by the screw hole, the advancing/retreating ball screw 664, the motor 25a and the like. Accordingly, the longitudinal movement drive unit 25 advances and retreats the frame 650 by rotationally driving the motor 25a, and thereby, can move the rotation side optical fiber FB1, the torque transmission coil 624, the fixed member 626 and the optical lens 628 in the directions of S1 and S2 of FIG. 4.

The motor 25a performs advance/retreat drive at a predetermined pitch speed, for example, 0.5 mm/sec, and at each of the predetermined pitches, the motor 24a causes the rotation side optical fiber FB1, the torque transmission coil 624 and the optical lens 628 to make one rotation at, for example, 50 Hz (3000 rpm), whereby the measurement light L1 is irradiated to the measuring object S by radial scanning.

According to the configuration as above, the rotation side optical fiber FB1 and the torque transmission coil 624 are rotated in the arrow R direction in FIG. 3 by the optical rotary joint 18, and thereby, the OCT probe 600 irradiates the measuring object S with the measurement light L1 radiated from the optical lens 628 while performing radial scanning in the arrow R direction (circumferential direction of the sheath 620) and acquires the return light L3.

Thereby, in the whole circumference in the circumferential direction of the sheath 620, the desired part of the measuring object S can be accurately captured, and the return light L3 reflected by the measuring object S can be obtained.

Further, when a plurality of pieces of optical structure information for generating an optical three-dimensional structure image are to be obtained, the optical lens 628 is moved to the terminal end of the movable range in the arrow S1 direction by the longitudinal movement drive unit 25, is moved in the arrow S2 direction by a predetermined amount while acquiring the optical structure information constituted of tomographic images, or alternately repeating acquisition of the optical structure information and movement in the S2 direction by the predetermined amount, and is moved to the terminal end of the movable range.

As above, a plurality of kinds of optical structure information in the desired range are obtained for the measuring object S, and an optical three-dimensional image can be obtained based on a plurality of pieces of information which are acquired.

More specifically, the optical structure information in the depth direction (first direction) of the measuring object S is acquired by the coherent signal, and radial scanning is performed for the measuring object S in the arrow R direction (circumferential direction of the sheath 620) of FIG. 3, whereby the optical structure information on the scan surface composed of the depth direction (firs direction) of the measuring object S and the direction (second direction) substantially orthogonal to the depth direction can be acquired. Further, by moving the scan surface along the direction (third direction) substantially orthogonal to the scan surface, a plurality of pieces of optical structure information for generating the optical three-dimensional structure image can be acquired.

FIG. 5 is a view showing the state of obtaining the optical structure information by using the OCT probe which is led out from the forceps channel of the endoscope of FIG. 1. As shown in FIG. 5, the distal end part of the insertion part 602 of the OCT probe is moved close to the desired part of the measuring object S, and the optical structure information is obtained. When a plurality of pieces of optical structure information in the desired range are to be acquired, the main body of the OCT probe 600 does not have to be moved, but the optical lens 628 only has to be moved within the sheath 620 by the advance/retreat drive part of the aforementioned optical rotary joint 18.

[Signal Processing Unit]

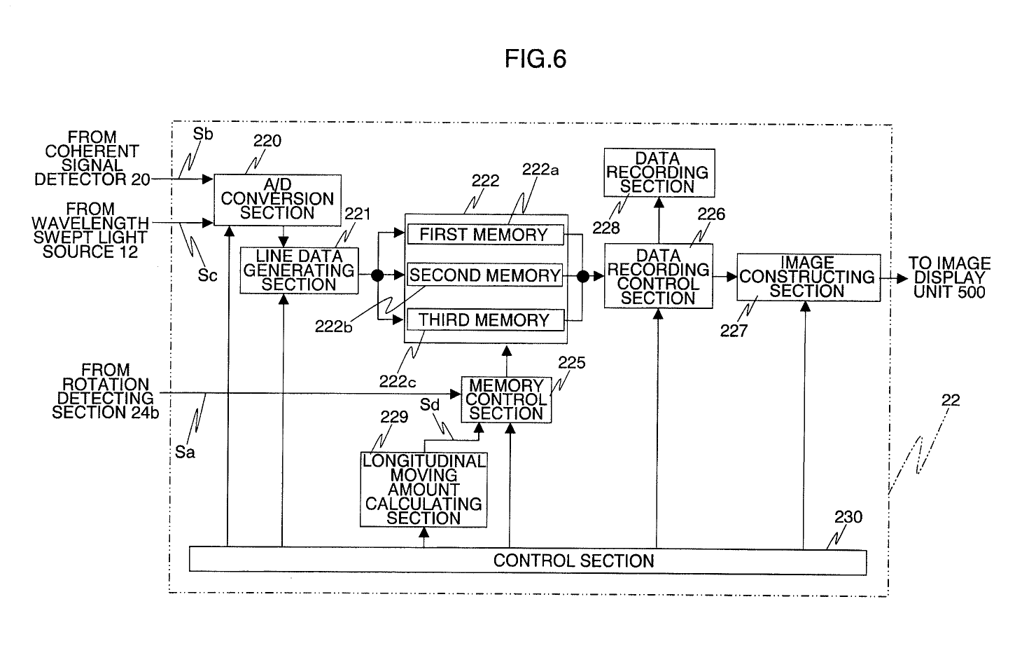

FIG. 6 is a block diagram showing the configuration of the signal processing unit of FIG. 2.

As shown in FIG. 6, the signal processing unit 22 is configured by including an A/D conversion section 220, a line data generating section 221 as a tomographic information generating device, a frame memory section 222 as a tomographic information storing device, a memory control section 225 as a storage control device and an evenly spaced tomographic image generating device, a data recording control section 226, an image constructing section 227 as a three-dimensional image generating device, a data recording section 228, an longitudinal moving amount calculating section 229 as a moving distance signal output device and a control section 230. The control section 230 controls the above described respective sections in the signal processing unit 22.

The A/D conversion section 220 converts a coherent signal of each radial scanning line from the coherent signal detecting unit 20 into a digital signal.

In detail, the A/D conversion section 220 performs A/D conversion of a coherent signal with the wavelength sweep synchronizing signal Sc which is outputted to be synchronized with the period of wavelength sweep from the wavelength swept light source 12 as a trigger. As a result, the data corresponding to wavelength sweep of one time becomes the coherent signal of one digitized radial scanning line.

The line data generating section 221 executes fast Fourier transform (FFT) processing for the coherent signal of each radial scanning line which is digitized in the A/D conversion section 220 to perform frequency decomposition to set the result as reflection intensity data in the depth direction of the measuring object S, performs logarithm transform of the data, and outputs the data to the frame memory section 222.

The frame memory section 222 stores the reflection intensity data from the line data generating section 221 based on the rotation detection signal Sa by frame unit, and is configured by including a first memory 222a, a second memory 222b and a third memory 222c which are constituted of three frame memories for storing the reflection intensity data of three frames, for example.

The memory control section 225 controls write of the reflection intensity data to the first memory 222a, the second memory 222b and the third memory 222c in the frame memory section 222 based on the rotation detection signal Sa, and controls read of the reflection intensity data from the first memory 222a, the second memory 222b and the third memory 222c based on a moving distance conversion signal Sd from the longitudinal moving amount calculating section 229.

The data recording control section 226 controls recording of the reflection intensity data of each radial scanning line stored in the frame memory section 222 into the data recording section 228.

The image constructing section 227 performs brightness control, contrast control, gamma correction, resampling corresponding to a display size, coordinates conversion corresponding to a scanning method and the like for the reflection intensity data of each radial scanning line via the data recording control section 226, generates a tomographic image of one frame, and displays the tomographic image on the image display unit 500.

The data recording section 228 stores the reflection intensity data of each radial scanning line stored in the frame memory section 222. The data recording section 228 is configured by, for example, a hard disk, a DVD disk, a blue ray disk, a semiconductor memory capable of reading/writing, or the like.

The longitudinal moving amount calculating section 229 outputs a pulse as the moving distance conversion signal Sd to the memory control section 225 at a time interval at which a tomographic image of each frame is acquired in the longitudinal moving speed which is set (by the longitudinal movement drive section 25 (see FIG. 4) which is configured by the screw hole, the advancing/retreating ball screw 664, the motor 660 and the like). For example, when the rotational speed of radial scanning is set as 50 Hz, and the longitudinal scanning speed is set as 0.5 mm/sec, pulses are outputted to the frame memory section 222 as the moving distance conversion signal Sd at intervals of 20 μsec ( 1/50 msec). The moving distance conversion signal Sd becomes a pulse at an interval of 10 μm when the signal is converted into the moving amount of the longitudinal movement drive section 25.

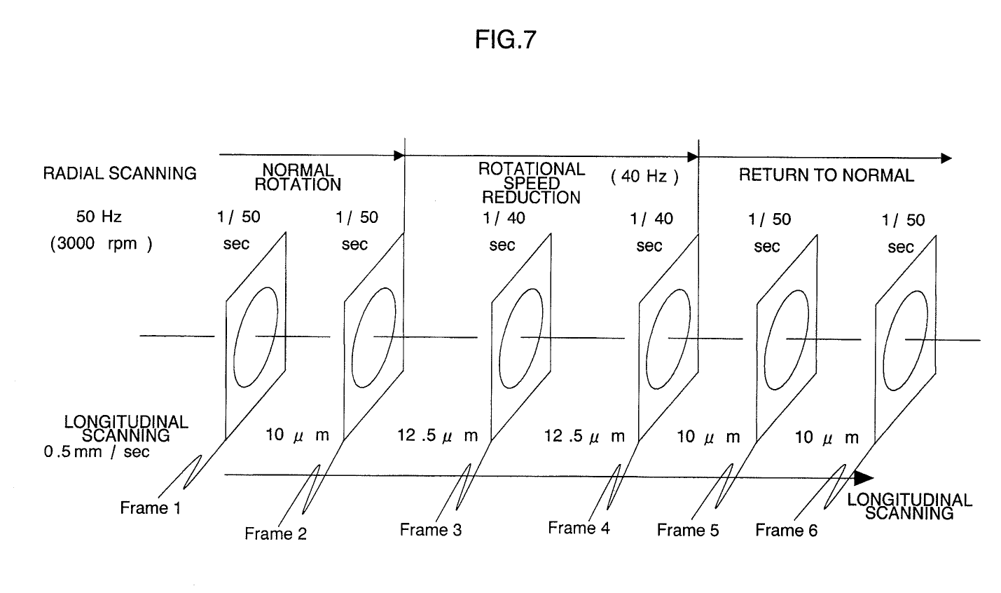

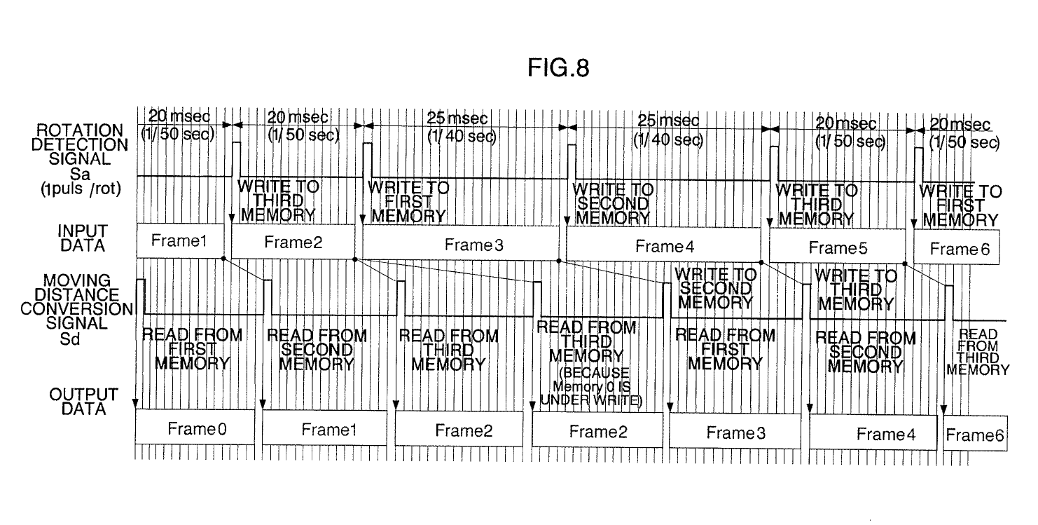

Here, general operations of the frame memory section 222 and the data recording control section 226 which are the essential parts of the present invention will be described. FIGS. 7 to 9 are diagrams for explaining the general operations of the frame memory section and the data recording control section of FIG. 6. As shown in FIG. 7, when the rotational speed of the torque transmission coil 624 temporarily reduces to 40 Hz from 50 Hz, for example, the reflection intensity data [Frame 1], [Frame 2], [Frame 3], . . . which are outputted from the line data generating section 221 are written to the frame memory section 222 as the input data by frame unit based on the rotation detection signal Sa (pulse rise timing) as shown in FIG. 8.

Meanwhile, the reflection intensity data [Frame 1], [Frame 2], [Frame 3], . . . by frame unit which are written to the frame memory section 222 are read as the output data by frame unit based on the moving distance conversion signal Sd (pulse rise timing) by control of the data recording control section 226, and is outputted to the data recording control section 226 at the rear stage.

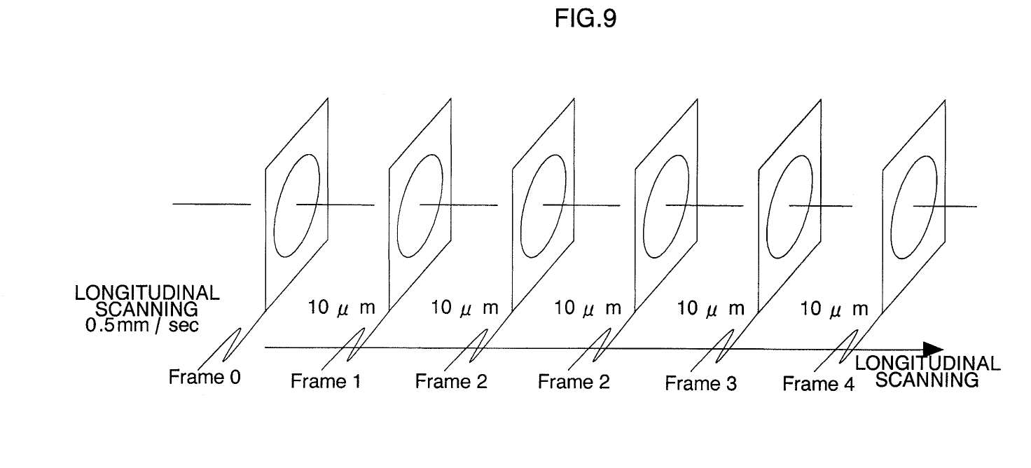

For example, in the case of FIG. 7, even when the rotational speed temporarily reduces to 40 Hz from 50 Hz during acquiring data of [Frame 3], data of [Frame 2] is read twice from the frame memory section 222 by control of the data recording control section 226 as shown in FIG. 8, and thereby, reduction in precision due to a variation of the rotational speed can be reduced to the minimum as shown in FIG. 9.

Though not illustrated, when rotation of radial scanning becomes temporarily high on the contrary, the reflection intensity data, which is written to the frame memory section 222 but is not read, occurs, and by thinning out unnecessary data as a result, reduction in precision can be similarly suppressed to the minimum.

As understood from this operation, if the interval of the moving distance conversion signal Sd is properly set, the reflection intensity data is interpolated or thinned out by control of the data recording control section 226 in correspondence with the interval, and the reflection intensity data can be reconstructed within a constant precision when seen as a whole.

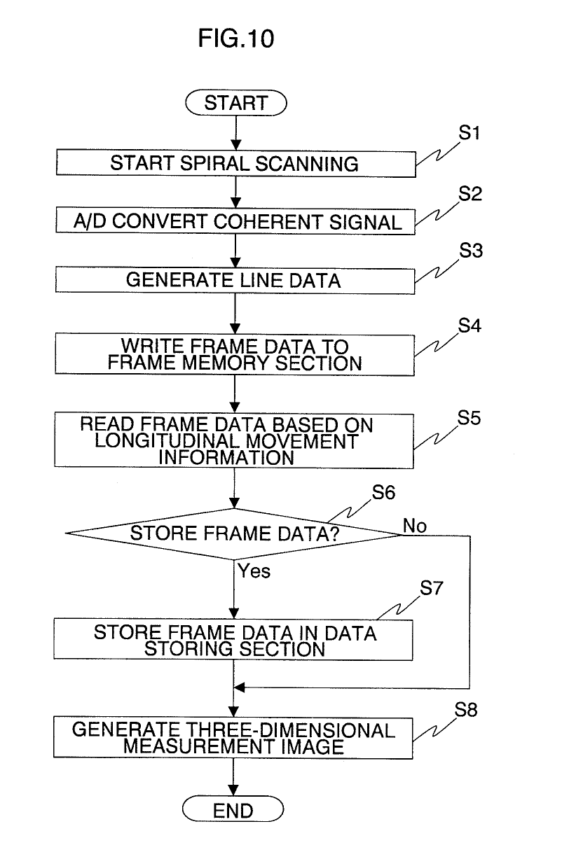

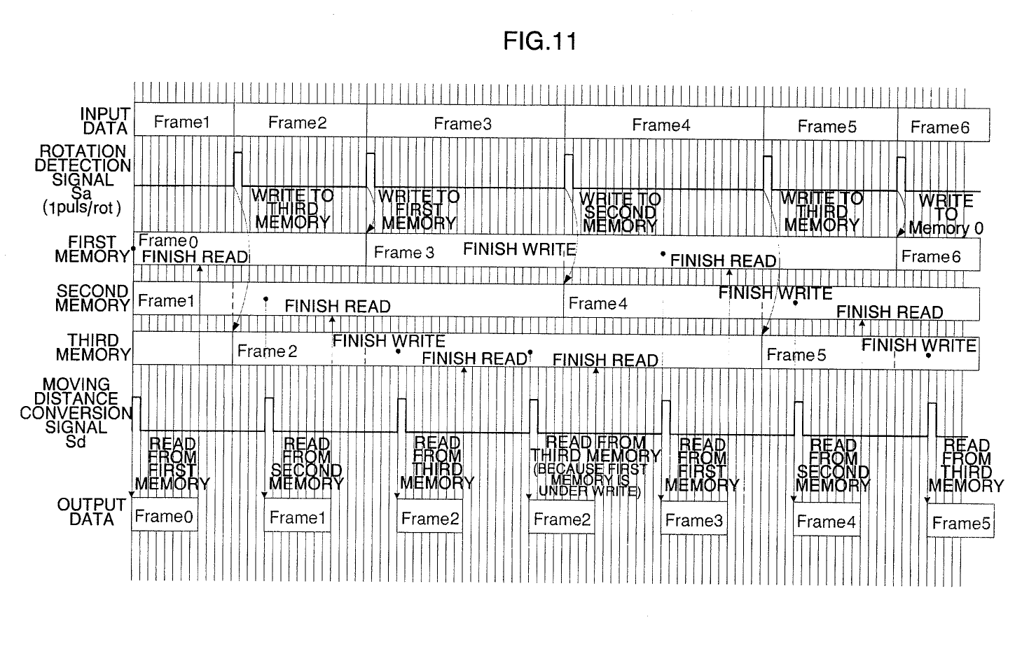

The operation of the present embodiment thus configured will be described by using FIGS. 10 and 11. FIG. 10 is a flowchart showing a flow of a process of the signal processing unit of FIG. 6, and FIG. 11 is a timing chart showing a timing of a signal of the frame memory section in the process of FIG. 10.

First, an operator turns on the power supply of the endoscope 100, the endoscope processor 200, the light source device 300, the OCT processor 400 and the image display unit 500 which configure the image diagnostic apparatus 10, inserts the insertion part 114 of the endoscope 100 into a body cavity, and moves the distal end part 144 of the endoscope 100 close to the measuring object S in the body cavity. Subsequently, the operator causes the distal end of the OCT probe 600 to abut on the measuring object S.

In this state, as shown in FIG. 10, the OCT probe 600 starts radial scanning of the measurement light L1 for the measuring object S (step S1).

Subsequently, the signal processing unit 22 performs A/D conversion of the coherent signal with the wavelength sweep synchronizing signal Sc which is outputted to be synchronized with the period of the wavelength sweep from the wavelength swept light source 12 as a trigger in the A/D conversion section 220. As a result, the data corresponding to the wavelength sweep of one time becomes the coherent signal of one radial scanning line which is digitized (step S2). Next, the signal processing unit 22 executes fast Fourier transform (FFT) processing for the coherent signal of each radial scanning line which is digitized in the A/D conversion section 220 and performs frequency decomposition, sets the result as the reflection intensity data in the depth direction of the measuring object S, performs logarithm transform of the data, and outputs the data to the frame memory section 222 in the line data generating section 221 (step S3).

Thereafter, the signal processing unit 22 causes the frame memory section 222 to store the reflection intensity data from the line data generating section 221 by frame unit based on the rotation detection signal Sa by control of the memory control section 225 (step S4).

The frame memory section 222 is configured by the first memory 222a, the second memory 222b and the third memory 222c of the frame memories of three frames as described above. In step S4, the reflection intensity data outputted from the line data generating section 221 are written to the first memory 222a, the second memory 222b and the third memory 222c which are frame memories based on the rotation detection signal Sa as described above, and at this time, the reflection intensity data is recorded in the frame memory having older stored reflection intensity data among the frame memories for which read processing is not performed.

Explaining with the case in which the input data is the reflection intensity data of [Frame 4] in FIG. 11, when the reflection intensity data of [Frame 4] is inputted, the respective frame memories store

the first memory 222a=the reflection intensity data of [Frame 3],

the second memory 222b=the reflection intensity data of [Frame 1], and

the third memory 222c=the reflection intensity data of [Frame 2 (under read)].

In this case, when the first memory 222a and the second memory 222b which are not under read are seen, the reflection intensity data recorded in the second memory 222b is older reflection intensity data (Frame 1), and therefore, the reflection intensity data of [Frame 4] is written to the second memory 222b.

Returning to FIG. 10, the signal processing unit 22 reads the reflection intensity data from the first memory 222a, the second memory 222b and the third memory 222c based on the moving distance conversion signal Sd from the longitudinal moving amount calculating section 229 by control of the memory control section 225 (step S5).

Read of the reflection intensity data in step S5 is executed based on the moving distance conversion signal, and at this time, reflection intensity data is read from the frame memory which has newer stored data among the frame memories in which write processing is not performed.

Describing with the case in which the output data is the reflection intensity data of [Frame 3] in FIG. 11, when Frame 3 is to be read, the respective frame memories store

the first memory 222a=the reflection intensity data of [Frame 3],

the second memory 222b=the reflection intensity data of [Frame 4 (under write)], and

the third memory 222c=the reflection intensity data of [Frame 2]. Therefore, when the first memory 222a and the third memory 222c which are not under write are seen, the first memory 222a stores newer reflection intensity data (Frame 3). Therefore, the reflection intensity data is read from the first memory 222a.

Here, the memories for three frames are adopted, but the memories are not especially limited to this value, and the similar effect can be obtained with the memories for four frames or more. Further, the memories for two frames can be realized, but in this case, if at the timing at which write of one frame is finished, the other memory is under read, write is performed for the same memory again, and the same data is repeatedly outputted accordingly. Thus, the precision of data reduces when seen as a whole.

Returning to FIG. 10, the signal processing unit 22 outputs the reflection intensity data from the frame memory section 22 to the data recording control section 226 to determine whether or not the reflection intensity data is recorded (stored) in the data recording section 228.

When recording the reflection intensity data is needed, the data is recorded in the data recording section 228 such as a hard disk, a DVD disk or the like in the data recording control section 226 (step S7).

Whether to record the data or not is set by being inputted from a user interface (not illustrated). Based on the control signal from the control section 230, the data recording control section 226 is controlled. The reflection intensity data outputted from the data recording control section 226 is inputted in the image constructing section 227.

Subsequently, the signal processing unit 22 performs brightness control, contrast control, gamma correction, resampling corresponding to the display size, coordinates conversion corresponding to the scanning method and the like for the reflection intensity data of each radial scanning line which goes through the data recording control section 226, generates a tomographic image of one frame in the image constructing section 227, and displays the three-dimensional measurement image on the image display unit 500 based on this tomographic image (step S8).

Like this, in the present embodiment, even when the rotational speed of radial scanning varies, reduction in the precision in the longitudinal direction is minimized, and the three-dimensional data (a plurality of tomographic images) can be acquired by longitudinal scanning. Thus, especially in real time during three-dimensional data OCT measurement, the three-dimensional measurement image can be constructed with reduction in precision of the evenly spaced tomographic images in the longitudinal direction being minimized.

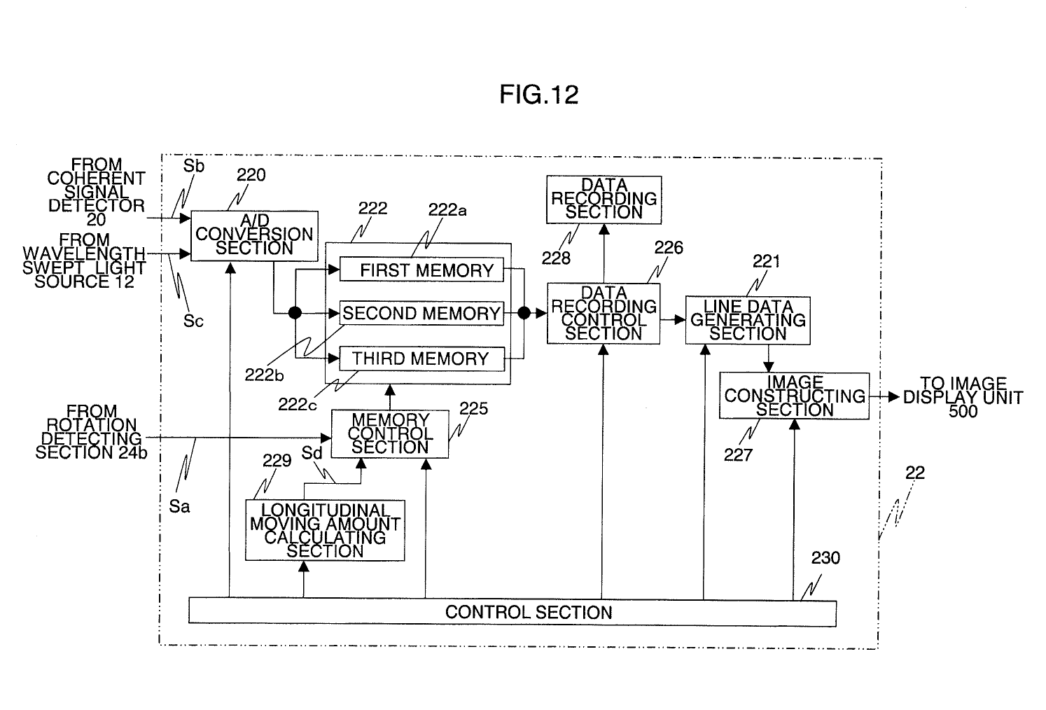

In the block configuration of the signal processing unit 22 shown in FIG. 6, the reflection intensity data from the line data generating section 221 is inputted in the frame memory section 222, but this is not restrictive. FIG. 12 is a diagram showing a modified example of the signal processing unit of FIG. 6. For example, as the block configuration of the signal processing unit 22, as shown in FIG. 12, the digitized coherent waveform data which is subjected to A/D conversion in the A/D conversion section 220 may be configured to be inputted in the frame memory section 222. In such a case, the coherent signal before FFT is performed is inputted in the frame memory section 222.

The data after FFT which becomes necessary here is the data through Nyquist frequency data, namely, only a half of the data at the low frequency side is required, and therefore, the capacity of the frame memories (the first memory 222a, the second memory 222b and the third memory 222c) can be made smaller when the line data generating section 221 is disposed ahead of the frame memory section 222.

### Second Embodiment

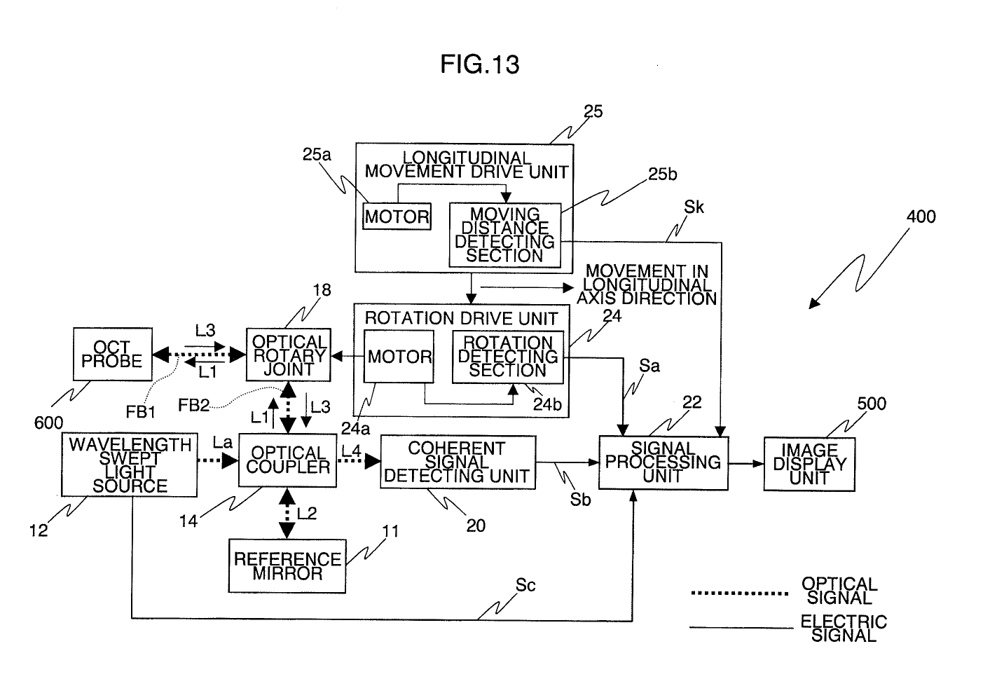

Next, a second embodiment of the present invention will be described. FIG. 13 is a block diagram showing a configuration of an OCT processor according to the second embodiment of the present invention. The second embodiment is substantially the same as the first embodiment, and therefore, only a different configuration will be described. The same configurations are assigned with the same reference numerals and characters, and the description of them will be omitted.

As shown in FIG. 13, the longitudinal movement drive section 25 of the OCT processor 400 of the present embodiment is configured by including a moving distance detecting section 25b as a moving distance signal outputting device which detects the linear movement in the longitudinal axis direction, and outputs an longitudinal moving distance detection signal Sk to the signal processing unit 22 at each movement of a constant distance in addition to the motor 25a which drives for longitudinal scanning.

As the longitudinal moving distance detection signal Sk which is outputted here, a pulse is desirably outputted at a distance interval at which a tomographic image of each frame is acquired. For example, when the rotational speed of radial scanning is set as 50 Hz (50 frame/sec), and the longitudinal scanning speed is set as 0.5 mm/sec, pulses are outputted at intervals of 10 μm.

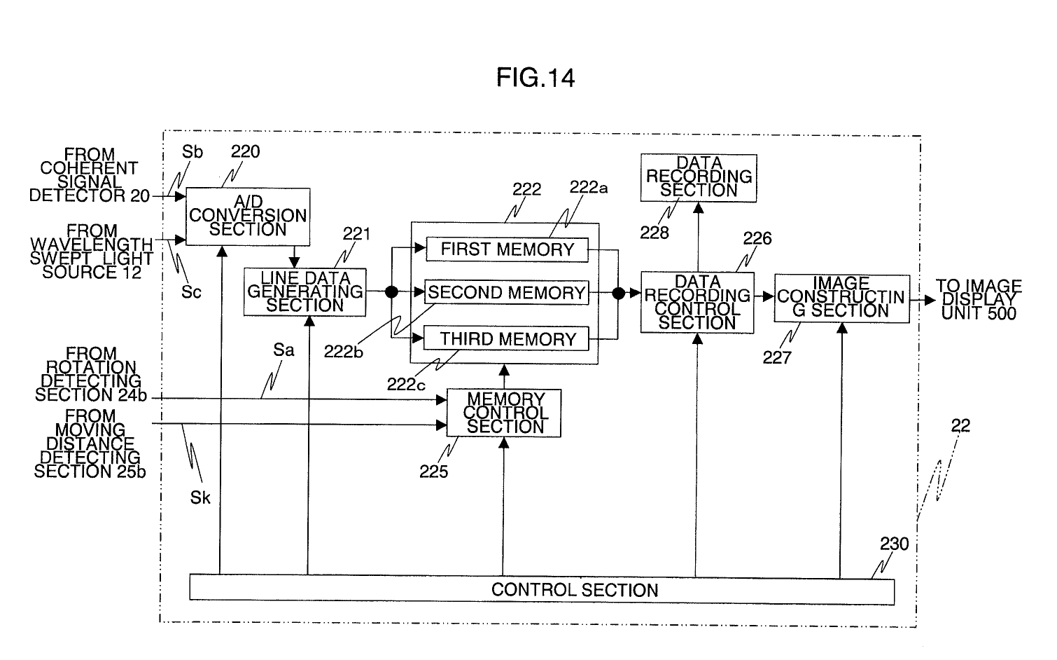

FIG. 14 is a block diagram of a signal processing unit of FIG. 13. What differs from the first embodiment here is that the longitudinal moving amount calculating section 229 (see FIG. 6) is absent, and a reading operation from the frame memory section 222 is performed based on the longitudinal moving distance detection signal Sk outputted from the moving distance detecting section 25b in place of the longitudinal moving distance conversion signal Sd in the first embodiment.

The other configurations and operations are the same as those in the first embodiment.

In the first embodiment, on the precondition that operation is performed so that the moving distance in the longitudinal direction is as set, the moving distance is estimated by time according to the longitudinal direction moving distance conversion signal Sd, and read from the frame memory section 222 is controlled, whereas in the second embodiment, the reading operation from the frame memory section 222 is controlled based on the actual moving distance according to the longitudinal moving distance detection signal Sk. Accordingly, in the second embodiment, three-dimensional image data with higher precision can be constructed as compared with the first embodiment, in addition to the operation and effect of the first embodiment.

### Third Embodiment

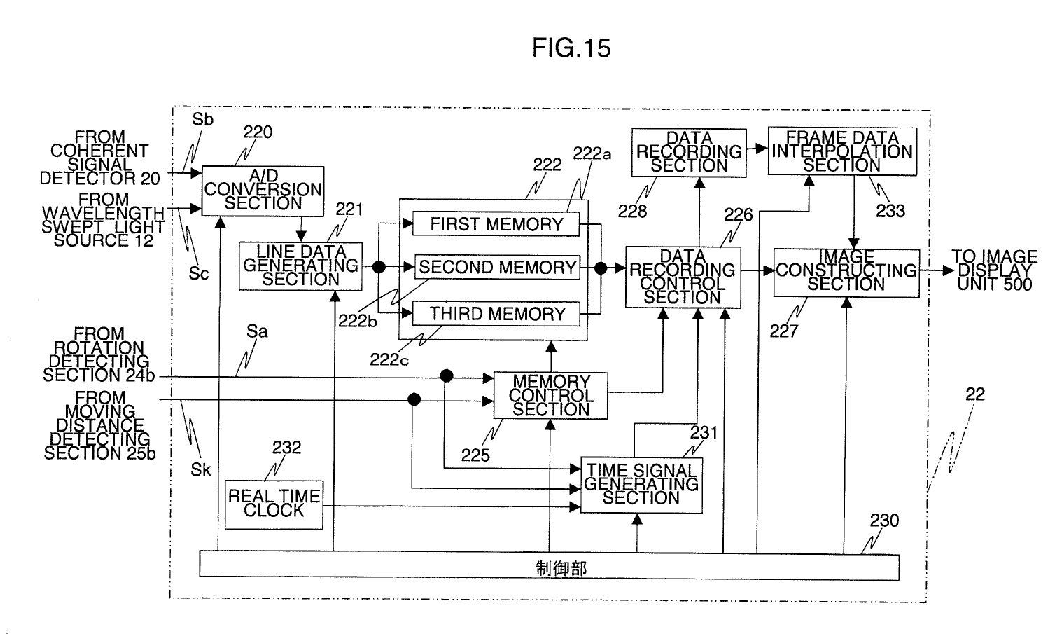

Next, a third embodiment of the present invention will be described. FIG. 15 is a block diagram of a signal processing unit according to the third embodiment of the present invention. The third embodiment is substantially the same as the second embodiment, and therefore, only a different configuration will be described. The same configurations are assigned with the same reference numerals and characters, and the description of them will be omitted.

In addition to the configuration of the second embodiment, the signal processing unit 22 of the present embodiment is configured by including a time signal generating section 231 as a time detecting device, a real time clock 232 and a frame data interpolation section 223 as a tomographic image interpolating and generating device.

The real time clock 232 is a clock which outputs an absolute time and is connected to the time signal generating section 231. The time signal generating section 231 outputs the absolute times, at which the rotation detection signal Sa outputted from the rotation detecting section 24b and the longitudinal moving distance detection signal Sk outputted from the longitudinal moving distance detecting section 25b are inputted, to the data recording control section 226. Further, the frame data interpolation section 223 generates the interpolated frame data which is the result of interpolating the reflection intensity data recorded in the data recording section 228, based on the absolute times at which the rotation detection signal Sa and the longitudinal moving distance detection signal Sk outputted from the longitudinal moving distance detecting section 25b are inputted. The other configurations are the same as those in the second embodiment.

A linking device is configured by the data recording control section 226, and a time-added tomographic information storing device is configured by the data recording section 228.

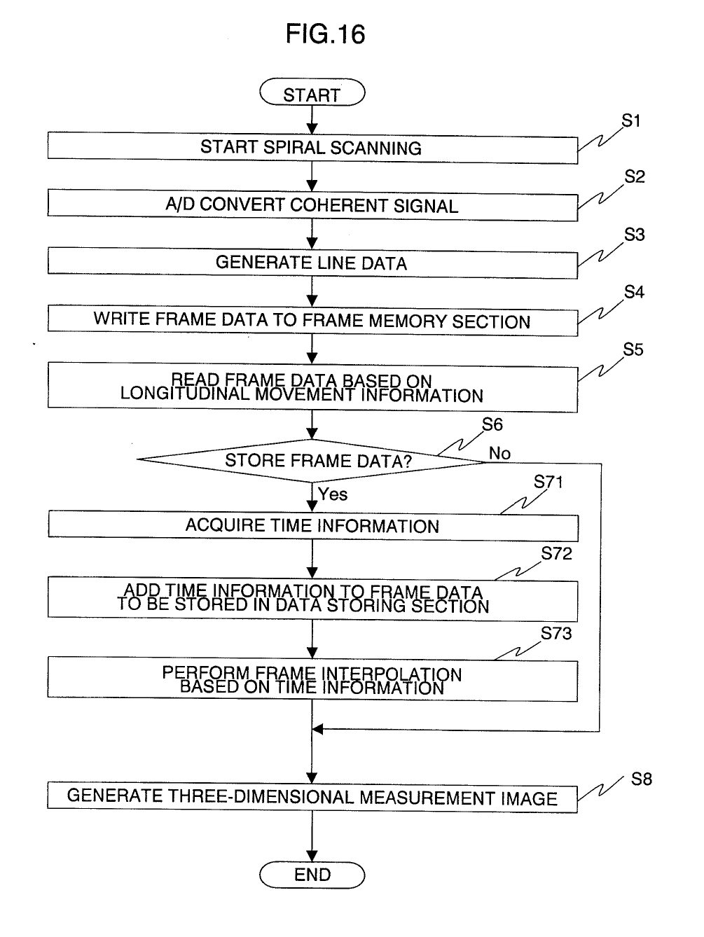

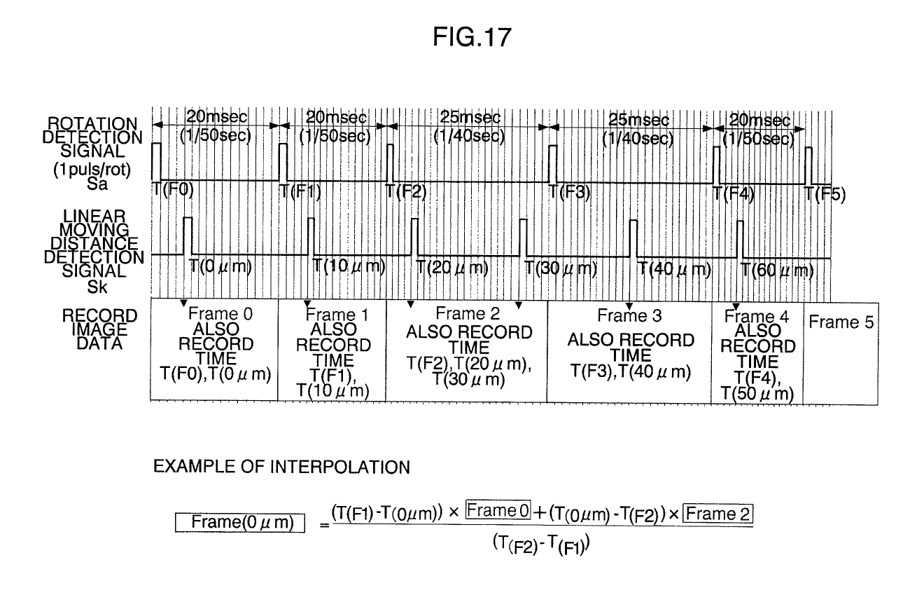

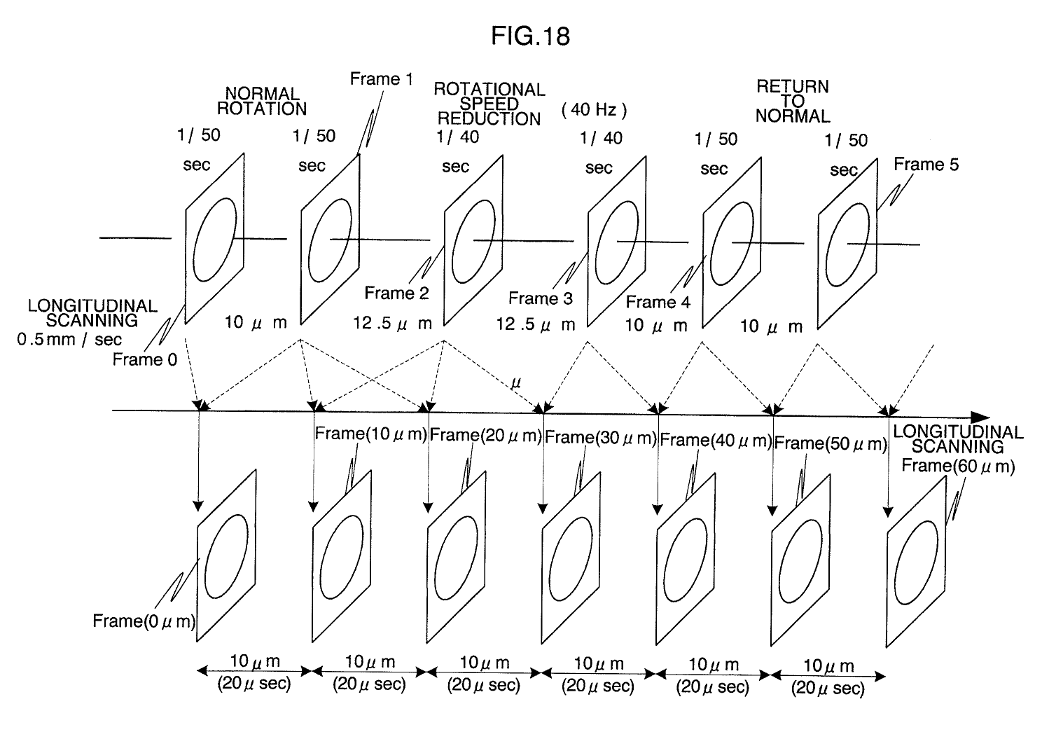

An operation of the present embodiment thus configured will be described by using FIGS. 16 to 18. FIG. 16 is a flowchart showing a flow of a process of the signal processing unit of FIG. 15. FIG. 17 is a timing chart showing a timing of a signal of the frame memory section in the process of FIG. 16. FIG. 18 is a diagram explaining the processing result of FIG. 16.

As shown in FIG. 16, the third embodiment differs from the second embodiment in that the processing of steps S71, S72 and S73 is performed instead of the processing of step S7 (see FIG. 10) described in the first embodiment.

More specifically, after the processing of steps S1 to S6 described in FIG. 10, the signal processing unit 22 acquires the absolute times at which the rotation detection signal Sa and the longitudinal moving distance detection signal Sk are inputted from the real time clock 232 in the time signal generating section 231, and outputs the absolute times to the data recording control section 226 (step S71).

Subsequently, the signal processing unit 22 adds the absolute times at which the rotation detection signal Sa and the longitudinal moving distance detection signal Sk are inputted to the reflection intensity data from the line data generating section 221 in the data recording control section 226, and stores the reflection intensity data in the data recording section 228 (step S72).

Describing the timing of data recording into the data recording section 228 by using FIG. 17, data recording into the data recording section 228 is performed by frame unit, and at this time, the absolute time data which is outputted from the time signal generating section 231 is recorded together as the header information of the frame data (reflection intensity data). Here, the absolute time data is recorded as the header information, but the method is not especially limited to this method, but any method may be adopted as long as the absolute time data is recorded by being linked to the reflection data. For example, the absolute time data may be similarly recorded as footer information, or the absolute time data may be stored as a separate file linked to the frame data (reflection intensity data).

Returning to FIG. 16, the signal processing unit 22 outputs the frame data (reflection intensity data) with the absolute time data being added which is recorded in the data recording section 228 to the frame data interpolation section 223 based on control from the data recording control section 226, and generates the data corresponding to the time of the longitudinal moving distance detection signal Sk from the frame data (reflection intensity data) and the absolute time data recorded in the frame data interpolation section 223 by interpolation from the frame data (reflection intensity data) before and after the data (step S73).

As the interpolation method in the frame data interpolation section 223, linear interpolation shown in the equation in the lower part of FIG. 17 is performed. Here, linear interpolation is adopted, but any method may be adopted such as B spline interpolation. The generated interpolated frame data is outputted to the image constructing section 227.

As a result, in the present embodiment, data can be generated at required longitudinal axis frame intervals after OCT measurement, for example, as shown in FIG. 18, in addition to the effect of the first and the second embodiments that “even when the rotational speed of radial scanning varies, three-dimensional data (a plurality of tomographic images) can be acquired by longitudinal scanning with reduction in precision in the longitudinal direction being minimized, and especially in real time during three-dimensional data OCT measurement, a three-dimensional measurement image can be constructed with reduction in the precision of the evenly spaced tomographic images in the longitudinal direction being minimized”. Therefore, even when the rotational speed of radial scanning varies, three-dimensional data can be acquired with reduction in the precision in the longitudinal direction being minimized.

Further, in the block configuration of the signal processing unit of FIG. 15, the reflection intensity data which is subjected to FFT is recorded in the data recording control section 226, but the line data generating section 221 may be disposed behind the data recording control section 226. In that case, reflection intensity data is generated in the line data generating section 221 while time information is held, and interpolation processing is performed in the frame data interpolation section 223.

Further, in the block configuration of the signal processing unit of FIG. 15, one system configuration is formed as a whole, but this may be divided into two systems. For example, the method may be adopted, which makes the frame data interpolation section 223 independent and one system. This is because if a product for general use purpose such as a hard disk or a DVD disk is adopted as the data recording section 228, the frame data interpolation section 223 can be configured by only an ordinary PC.

### Fourth Embodiment

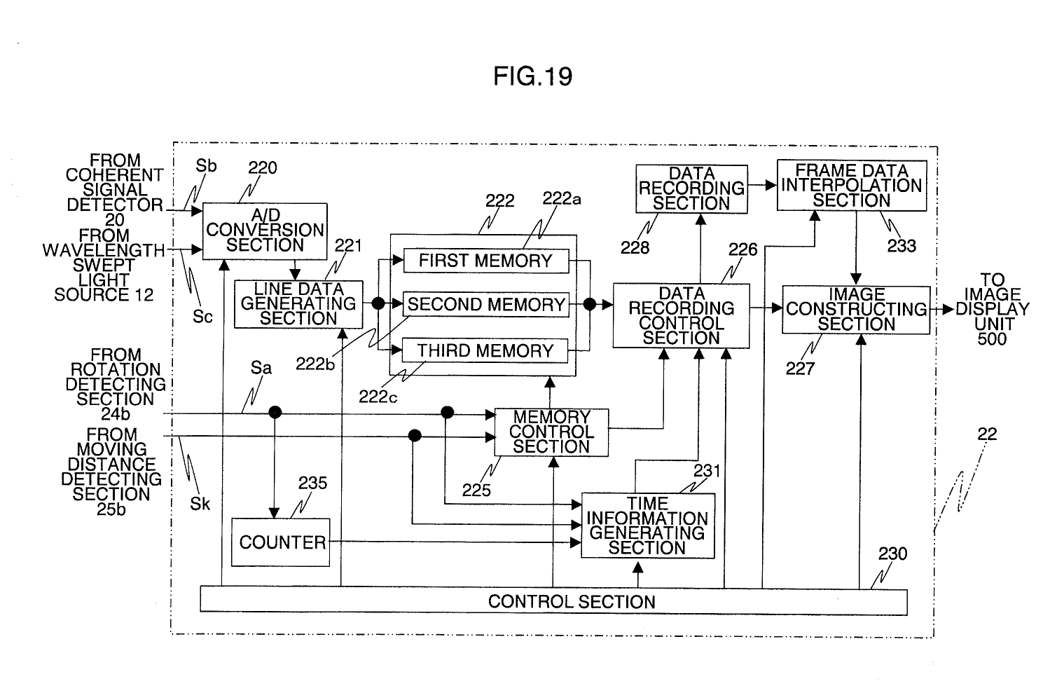

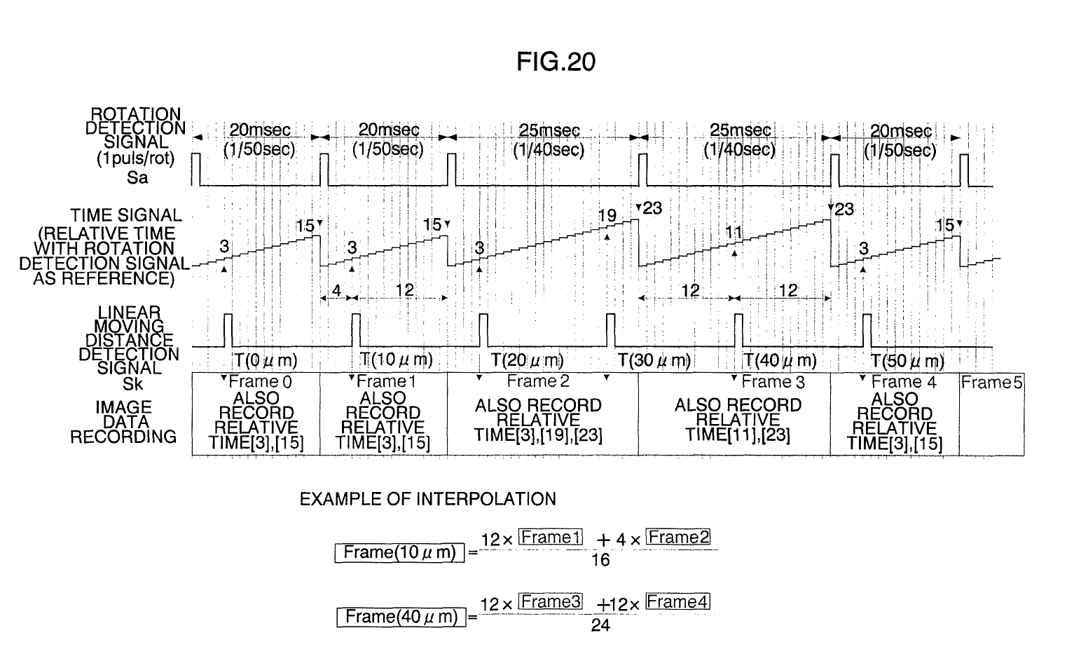

Next, a fourth embodiment of the present invention will be described. FIG. 19 is a block diagram of a signal processing unit according to the fourth embodiment of the present invention. FIG. 20 is a timing chart showing a timing of a signal of a frame memory section in a process of FIG. 19. The fourth embodiment is substantially the same as the third embodiment, and therefore, only a different configuration will be described. The same configurations are assigned with the same reference numerals and characters, and description of them will be omitted.

What differs from the third embodiment is the operation of the time signal generating section 231. The fourth embodiment has a counter 235 instead of the real time clock 232. The counter 235 may be provided inside the time signal generating section 231. The other components are the same as those in the third embodiment.

Describing the timing of data recording to the data recording section 228 in the present embodiment by using FIG. 20, the count value of the counter 235 is reset each time the rotation detection signal Sa is inputted, and count is started in an internal clock of the counter 235. The time signal generating section 231 outputs the count value of the counter 235 at the time when the linear moving distance output signal Sk is inputted, and the count value at the time when the rotation detection signal Sa which is a reset pulse is inputted to the data recording section. The following process is the same as that of the first embodiment.

As above, in the present embodiment, the effect similar to that of the third embodiment can be obtained.

In the above described first to fourth embodiments, the OCT processor 400 including the OCT probe 600 is described as the three-dimensional image constructing apparatus, but the three-dimensional image constructing apparatus of the present invention also can be applied to an ultrasound observation apparatus with ultrasound as a wave, and in the following fifth and sixth embodiments, the embodiments adopting the three-dimensional image constructing apparatuses as the ultrasound observation apparatus will be described.

### Fifth Embodiment

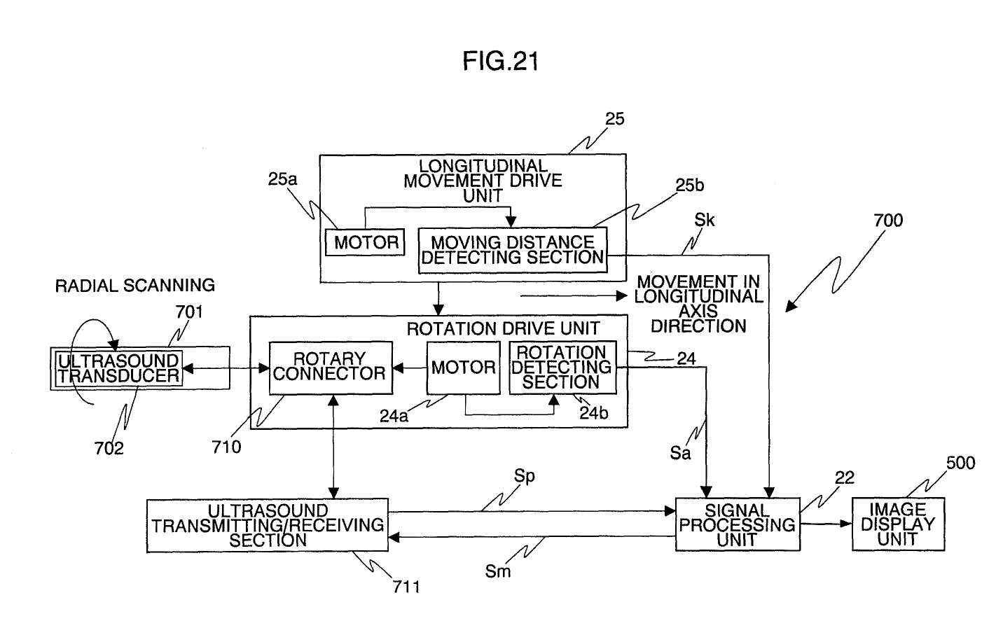

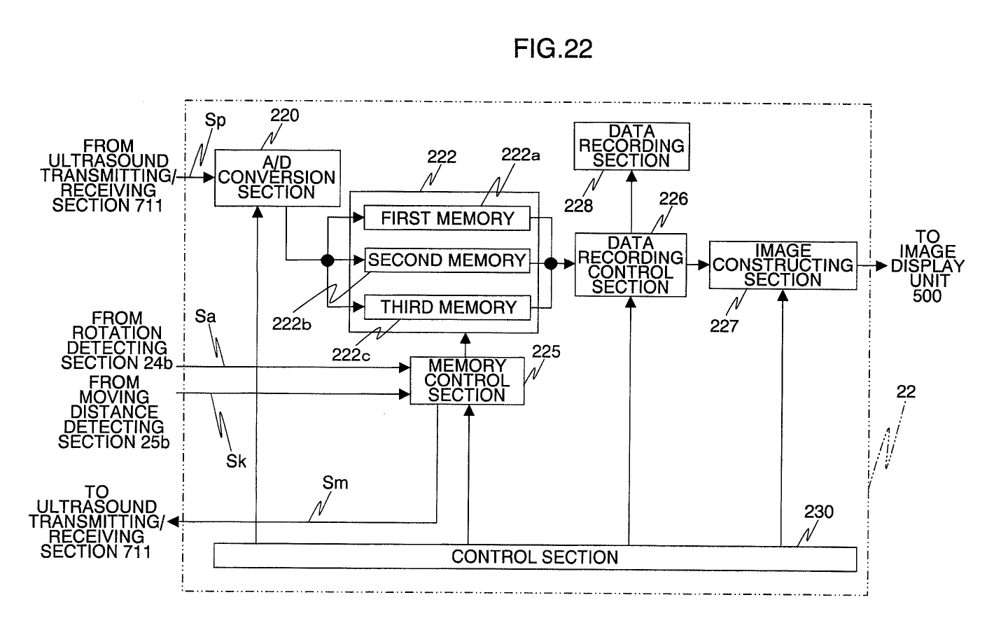

A fifth embodiment of the present invention will be described. FIG. 21 is a block diagram showing a configuration of an ultrasound observation apparatus according to the fifth embodiment of the present invention. FIG. 22 is a block diagram showing a configuration of a signal processing unit of FIG. 21. The configuration of the main part of the present embodiment is the same as the OCT processor described in the second embodiment, and therefore, only the different aspect will be described.

As shown in FIG. 21, in an ultrasound observation apparatus 700 of the present embodiment, a transmission trigger signal Sm which is outputted from the signal processing unit 22 is firstly inputted in an ultrasound signal transmitting/receiving unit 711, and based on the transmission trigger signal Sm, an ultrasound transmission signal is outputted to an ultrasound probe 701 through a rotary connector 710 from the ultrasound signal transmitting/receiving unit 711.

The ultrasound transmission signal is inputted in an ultrasound transducer 702 as a wave transmitting/receiving device disposed at a distal end of the ultrasound probe 701 which is rotatably connected by the rotary connector 710. In the ultrasound transducer 702, the inputted electric signal is converted into a mechanical vibration, and ultrasound as a wave is outputted to the measuring object S such as biological tissue. At this time, the ultrasound probe 701 is rotationally driven by the rotation drive unit 24, and performs radial scanning in a living body. Further, the rotation drive unit 24 is mechanically connected to the longitudinal movement drive unit 25, and the ultrasound probe 701 simultaneously moves in the longitudinal direction, and thereby, performs longitudinal scanning.

A reflective echo which is reflected by the measuring object S is converted into an electric signal from a mechanical vibration in the ultrasound transducer 702, and is inputted in the ultrasound signal transmitting/receiving unit 711 again as a reception echo signal Sp through the rotary connector 710. The reception echo signal Sp is subjected to filter processing, and analog signal processing such as gain control in the ultrasound signal transmitting/receiving unit 711, and thereafter, is inputted in the signal processing unit 22.

Further, the aforementioned rotation drive unit 24 is configured by the motor 24a for causing the ultrasound probe 701 to perform radial scanning and the rotation detecting section 24b that outputs a rotation signal. Two kinds of signals that are the pulses outputted at equal angle intervals per one rotation like 512 pulses/rotation, for example, and a signal that is outputted as one pulse per one rotation are outputted from the rotation detecting section 24b and are inputted in the signal processing unit 22. Here, 512 pulses/rotation are outputted, but the number of pulses is not especially limited to this value, and as the number is larger, scanning line density becomes higher, whereas as the number is smaller, the density becomes lower. Therefore, the value is determined by the balance of a resolution and a speed.

In the signal processing unit 22, the tomographic image of a living body is constructed by signal processing which will be described later, and is displayed on the image display unit 500 such as an LCD monitor.

Further, the longitudinal movement drive unit 25 is configured by the motor 25a which drives for longitudinal scanning and the moving distance detecting section 25b which detects movement in the linear direction and outputs an longitudinal moving distance detection signal at each movement by a fixed distance. The longitudinal moving distance detection signal Sk which is outputted here is outputted to the signal processing unit 22. As the longitudinal moving distance detection signal Sk, a pulse is desirably outputted at a distance interval at which a tomographic image of each frame is acquired. For example, when the rotational speed of radial scanning is set as 50 Hz (50 frame/sec) and the longitudinal scanning speed is set as 1 mm/sec, pulses are outputted at intervals of 20 μm.

Next, the configuration of the signal processing unit 22 of the present embodiment will be described. As shown in FIG. 22, the control section 230 performs centralized control of the entire signal processing unit 22.

The rotation detection signal Sa which is outputted from the aforementioned rotation detecting section 24b is inputted in the memory control section 225. Among them, based on the pulses outputted at equal angle intervals per one rotation, the memory control section 225 outputs the trigger signal Sm to the ultrasound transmitting/receiving section 711.

Meanwhile, the reception echo signal Sp which is outputted from the aforementioned ultrasound transmitting/receiving section 711 is inputted in the A/D conversion section 220, is subjected to A/D conversion and is converted into a digital signal. The digitized reception echo data is outputted to the frame memory section 222. The operation of the frame memory section 222 is the same as that of the second embodiment.

The digitized reception echo data which is outputted from the frame memory section 222 is outputted to the data recording control section 226, and when recording is necessary, the reception echo data is recorded in the data recording section 228 such as a hard disk and a DVD disk. Whether to record the data or not is set by being inputted from a user interface, and is controlled by the data recording control section 226 based on the control signal from the control section 230.

The reception echo data which is outputted from the data recording control section 226 is inputted in the image constructing section 227. In the image constructing section 227, wave detection processing, logarithm transform, brightness control, contrast control, gamma correction, resampling corresponding to a display size, coordinates conversion corresponding to a scanning method and the like are performed, and a tomographic image is generated.

As a result, in the present embodiment, even when the rotational speed of radial scanning varies, three-dimensional data also can be acquired by longitudinal scanning with reduction in precision in the longitudinal direction being minimized, as described in the first to the fourth embodiments.

### Sixth Embodiment

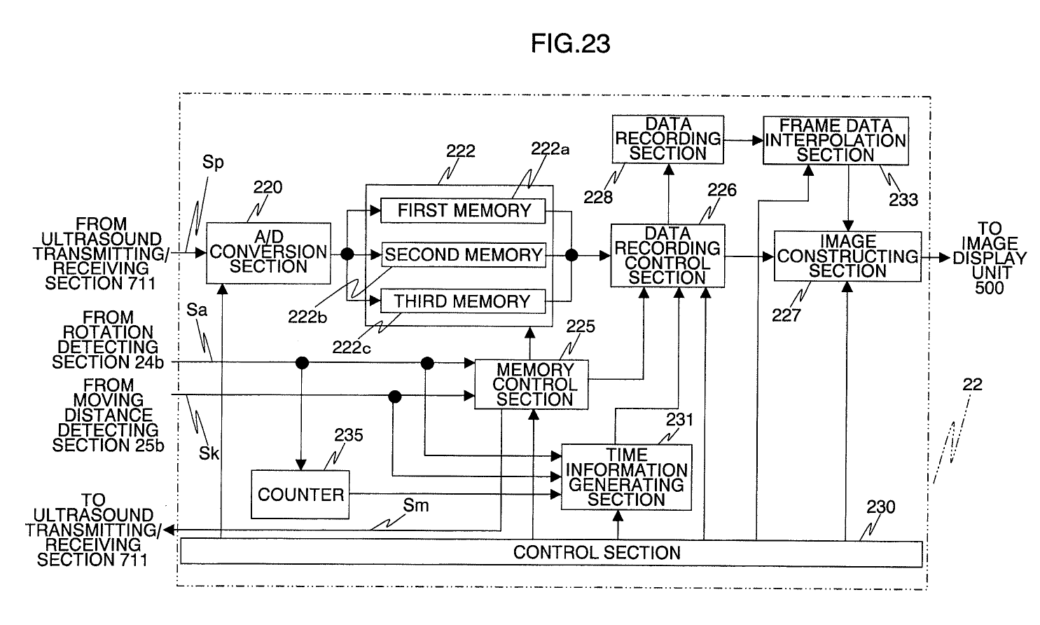

A sixth embodiment of the present invention will be described. FIG. 23 is a block diagram showing a configuration of a signal processing unit of an ultrasound observation apparatus according to the sixth embodiment of the present invention. The basic configuration of the present embodiment is substantially the same as that of the fifth embodiment, and the configuration of the main part is the same as that of the OCT processor described in the fourth embodiment. Therefore, only the different aspect will be described.

As shown in FIG. 23, in the signal processing unit 22 of an ultrasound observation apparatus of the present embodiment, the rotation detection signal Sa which is outputted from the rotation detecting section 24b (see FIG. 21) is inputted in the memory control section 225. Among them, based on the pulses outputted at equal angle intervals per one rotation, the trigger signal Sm is outputted to the ultrasound transmitting/receiving section 711 (see FIG. 21).

Meanwhile, the reception echo signal Sp outputted from the ultrasound transmitting/receiving section 711 is inputted in the A/D conversion section 220, is subjected to A/D conversion and is converted into a digital signal. The digitized reception echo data is outputted to the data recording control section 226.

In the data recording control section 226, the inputted reception echo data is recorded in the data recording section 228 such as a hard disk or a DVD disk when recording the inputted reception echo data is necessary. Reception echo data recording at this time is performed by frame unit, and the time data which is outputted from the time signal generating section 231 at this time is recorded together as header information of the frame data. Here, the time data is recorded as header information, but the method is not especially limited to this method, and any method may be adopted as long as the time data is recorded by being linked with the frame data. For example, the time data may be recorded similarly as footer information, or may be recorded as a separate file. The configuration and operation of the time signal generating section 231 are the same as those of the fourth embodiment.

The reception echo data recorded in the data recording section 228 is outputted to the frame data interpolation section 233 based on the control from the data recording control section 226. In the frame data interpolation section 233, from the recorded frame data (reception echo data) and the time data, the data corresponding to the time of the longitudinal moving distance detection signal Sk is generated by interpolation from the frame data before and after the data. The configuration and the operation of the frame data interpolation section 233 are the same as those of the fourth embodiment.

The interpolated frame data (reception echo data) which is generated in the frame data interpolation section 233 is outputted to the image constructing section 227. In the image constructing section 227, wave detection processing, logarithm transform, brightness control, contrast control, gamma correction, resampling corresponding to a display size, coordinates conversion corresponding to a scanning method and the like are performed, and a tomographic image is generated.

As a result, in the present embodiment, data is also generated at the frame intervals which are required, and even when the rotational speed of radial scanning varies, three-dimensional data also can be acquired with reduction in precision in the longitudinal direction being minimized, as described in the first to the fifth embodiments.

The three-dimensional image constructing apparatus of the present invention is described in detail above, but it goes without saying that the present invention is not limited to the above examples and various improvements and modifications may be made within the range without departing from the gist of the present invention.

What is claimed is:

1. A three-dimensional image constructing apparatus, comprising:
a wave transmitting/receiving device which is provided in a distal end of a slim and substantially tubular probe having flexibility, and transmits and receives a wave;
a transmission/reception wave rotating device which rotates the wave transmitting/receiving device around a longitudinal axis of the probe, and causes the wave to scan radially on a scan surface including a depth direction of a measuring object;
a rotation detecting device which detects rotation of the transmission/reception wave rotating device and outputs a rotation detection signal;
a tomographic information generating device which generates tomographic information of the measuring object from reflection wave information of the wave which is caused to scan radially and is reflected at the measuring object, based on the rotation detection signal from the rotation detecting device;
a tomographic information storing device which stores the tomographic information by frame unit;
a storage control device which controls write and read of tomographic information in the tomographic information storing device;
a transmission/reception wave moving device which moves the wave transmitting/receiving device along the longitudinal axis direction;
an evenly spaced tomographic image generating device which generates an evenly spaced tomographic image of the measuring object at a moving position at each of constant equal spaces along the longitudinal axis direction by the transmission/reception wave moving device, based on the tomographic information which is read from the tomographic information storing device by being controlled by the storage control device; and
a three-dimensional image generating device which generates a three-dimensional image of the measuring object based on the evenly spaced tomographic image.

2. The three-dimensional image constructing apparatus according to claim 1, further comprising: a first moving distance signal outputting device which estimates a moving distance of the wave transmitting/receiving device by the transmission/reception wave moving device in the longitudinal axis direction based on a time interval which is set in advance, and outputs a moving distance signal,
wherein the storage control device
writes the tomographic information into the tomographic information storing device synchronously with an output time of the rotation detection signal, and
reads the tomographic information stored in the tomographic information storing device synchronously with an output time of the moving distance signal.

3. The three-dimensional image constructing apparatus according to claim 1, further comprising: a second moving distance signal outputting device which detects a moving distance of the wave transmitting/receiving device in the longitudinal axis direction, and outputs a moving distance signal,
wherein the storage control device
writes the tomographic information into the tomographic information storing device synchronously with an output time of the rotation detection signal, and
reads the tomographic information stored in the tomographic information storing device synchronously with an output time of the moving distance signal.

4. The three-dimensional image constructing apparatus according to claim 1,
wherein the tomographic information storing device comprises a plurality of frame memories which store the tomographic information of a plurality of frames.

5. The three-dimensional image constructing apparatus according to claim 4,
wherein the storage control device stores the tomographic information which is newly generated by the tomographic information generating device in the frame memory which stores the earliest tomographic image in a sequence of generation by the tomographic information generating device, among the frame memories in which read processing is not performed in the tomographic information storing device, and reads the tomographic information from the frame memory which stores the latest tomographic information in the sequence of generation by the tomographic information generating device among the frame memories in which write processing is not performed in the tomographic information storing device.

6. The three-dimensional image constructing apparatus according to claim 4,
wherein the tomographic information storing device comprises at least three frame memories which store the tomographic information of at least three frames.

7. The three-dimensional image constructing apparatus according to claim 2, further comprising:
a time detecting device which detects a time of an output time of the rotation detection signal as first time information, and a time of an output time of the moving distance signal as second time information;
a linking device which links the tomographic information generated by the tomographic information generating device, and the first time information and the second time information; and
a time-added tomographic information storing device which stores the tomographic information to which the first time information and the second time information are linked in the linking device as time-added tomographic information.

8. The three-dimensional image constructing apparatus according to claim 3, further comprising:
a time detecting device which detects a time of an output time of the rotation detection signal as first time information, and a time of an output time of the moving distance signal as second time information;
a linking device which links the tomographic information generated by the tomographic information generating device, and the first time information and the second time information; and
a time-added tomographic information storing device which stores the tomographic information to which the first time information and the second time information are linked in the linking device as time-added tomographic information.

9. The three-dimensional image constructing apparatus according to claim 7, further comprising: a real time clock having absolute time information,
wherein the time detecting device detects the first time information and the second time information based on the absolute time information of the real time clock.

10. The three-dimensional image constructing apparatus according to claim 7,
wherein the time detecting device detects a relative time with a detection time of the first time information as a reference, as the second time information.

11. The three-dimensional image constructing apparatus according to claim 7, further comprising: a tomographic image interpolating and generating device which interpolates the tomographic information and generates the evenly spaced tomographic image, based on the first time information and the second time information in accordance with a plurality of pieces of time-added tomographic information stored in the time-added tomographic information storing device.

12. The three-dimensional image constructing apparatus according to claim 1, wherein the transmission/reception wave rotating device is a flexible shaft with the longitudinal axis provided in the probe including the wave transmitting/receiving device at a distal end as a rotation axis, and the transmission/reception wave moving device moves the flexible shaft along the longitudinal axis.

13. The three-dimensional image constructing apparatus according to claim 1,
wherein the wave is a light, and the light is divided into a measurement light and a reference light,
the probe is connected to a light source which outputs the light, through the optical rotary joint, and is capable of transmitting and receiving the measurement light; and
the tomographic information generating device generates the tomographic information by the frame unit based on a coherent light of a reflection light of the measurement light in a body cavity acquired by the probe and the reference light reflected in a predetermined path.

14. The three-dimensional image constructing apparatus according to claim 13,
wherein the light source is a wavelength swept laser source.

15. The three-dimensional image constructing apparatus according to claim 1,
wherein the wave is ultrasound,
the probe includes an ultrasound transducer capable of transmitting and receiving the ultrasound, and
the tomographic information generating device generates the tomographic information by the frame unit based on an echo signal of the ultrasound in the body cavity which is acquired by the probe.

16. An image processing method of a three-dimensional image constructing apparatus, comprising the steps of:
a transmission/reception wave rotating step of rotating a wave transmitting/receiving device, which is provided in a distal end of a slim and substantially tubular probe having flexibility and transmits and receives a wave, around a longitudinal axis of the probe, and causing the wave to scan radially on a scan surface including a depth direction of a measuring object;
a rotation detecting step of detecting rotation in the transmission/reception wave rotating step, and outputting a rotation detection signal;
a tomographic information generating step of generating tomographic information of the measuring object from reflection wave information of the wave which is caused to scan radially and reflected at the measuring object, based on the rotation detection signal from the rotation detecting step;
a tomographic information storing step of storing the tomographic information by frame unit;
a storage control step of controlling write and read of tomographic information in the tomographic information storing step;
a transmission/reception wave moving step of moving the wave transmitting/receiving device along the longitudinal axis direction;
an evenly spaced tomographic image generating step of generating an evenly spaced tomographic image of the measuring object at a moving position at each of constant equal spaces along the longitudinal axis direction by the transmission/reception wave moving step, based on the tomographic information which is read from a tomographic information storing step by being controlled by the storage control step; and
a three-dimensional image generating step of generating a three-dimensional image of the measuring object based on the evenly spaced tomographic image.

17. The image processing method of the three-dimensional image constructing apparatus according to claim 16, further comprising: a first moving distance signal outputting step of estimating a moving distance of the wave transmitting/receiving device by a transmission/reception wave moving step in the longitudinal axis direction based on a time interval which is set in advance, and outputting a moving distance signal,
wherein in the storage control step
the tomographic information is written in the tomographic information storing step synchronously with an output timing of the rotation detection signal, and
the tomographic information is read from the tomographic information storing step synchronously with an output timing of the moving distance signal.

18. The image processing method of the three-dimensional image constructing apparatus according to claim 16, further comprising: a second moving distance signal outputting step of detecting a moving distance in the wave transmitting/receiving moving step in the longitudinal axis direction, and outputting a moving distance signal,
wherein in the storage control step
the tomographic information is written in the tomographic information storing step synchronously with an output timing of the rotation detection signal, and
the tomographic information is read from the tomographic information storing step synchronously with an output timing of the moving distance signal.

19. The image processing method of the three-dimensional image constructing apparatus according to claim 17,
further comprising:
a time detecting step of detecting a time of an output time of the rotation detection signal as first time information, and a time of an output time of the moving distance signal as second time information;
a linking step of linking the tomographic information generated in the tomographic information generating step, and the first time information and the second time information; and
a time-added tomographic information storing step of storing the tomographic information to which the first time information and the second time information are linked in the linking step as time-added tomographic information.

20. The image processing method of the three-dimensional image constructing apparatus according to claim 18,
further comprising:
a time detecting step of detecting a time of an output time of the rotation detection signal as first time information, and a time of an output time of the moving distance signal as second time information;
a linking step of linking the tomographic information generated in the tomographic information generating step, and the first time information and the second time information; and
a time-added tomographic information storing step of storing the tomographic information to which the first time information and the second time information are linked in the linking step as time-added tomographic information.

21. The image processing method of the three-dimensional image constructing apparatus according to claim 19,
wherein in the time detecting step, the first time information and the second time information are detected based on absolute time information from the real time clock.

22. The image processing method of the three-dimensional image constructing apparatus according to claim 19,
wherein in the time detecting step, a relative time with a detection time of the first time information as a reference is detected as the second time information.

23. The image processing method of the three-dimensional image constructing apparatus according to claim 19, further comprising: a tomographic image interpolating and generating step of interpolating the tomographic information and generating the evenly spaced tomographic image, based on the first time information and the second time information in accordance with a plurality of pieces of time-added tomographic information stored in the time-added tomographic information storing step.
